(19) United States
(12) Patent Application Publication
(10) Pub. No.: US 2019/0315790 A1
(43) Pub. Date: Oct. 17, 2019

(54) SYNTHESIS OF D-ALLULOSE

(71) Applicant: PFEIFER & LANGEN GMBH & Co. KG, Köln (DE)

(72) Inventors: Timo Johannes Koch, Elsdorf (DE); Florian Kipping, Dormagen (DE); Steffen Butz, Kreuzau (DE); Marcel Lesch, Bergheim (DE); Thomas Hässler, Köln (DE)

(73) Assignee: PFEIFER & LANGEN GMBH & Co. KG, Köln (DE)

(21) Appl. No.: 16/346,848

(22) PCT Filed: Nov. 9, 2017
(86) PCT No.: PCT/EP2017/078819

§ 371 Date: May 1, 2019

(30) Foreign Application Priority Data
Nov. 11, 2016 (EP) 16198388.7

(51) Int. Cl. C07H 1/06; B01D 15/18; C07H 3/02; C12P 19/02

(52) U.S. Cl. CPC C07H 1/06; B01D 15/185; C07H 3/02; C12Y 501/03; C12Y 204/01007; C12Y 204/0102; C12P 19/02

(57) ABSTRACT

The invention relates to a process for the synthesis of a product saccharide, preferably of D-allulose from an educt saccharide, preferably from D-fructose under heterogeneous or homogeneous catalysis which includes chemical and/or enzymatic catalysis. The synthesis is performed in at least two reactors that are arranged in series and the reaction product exiting the first reactor is subjected to chromatographic separation before it enters the second reactor. Preferably, the chromatographic separation is integrated in a simulated moving bed.

The invention relates to a process for the synthesis of a product saccharide, preferably of D-allulose, from an educt saccharide, preferably from D-fructose, under heterogeneous or homogeneous catalysis which includes chemical and/or enzymatic catalysis and also for providing a solid product saccharide product, preferably a solid allulose product. The synthesis is performed in at least two reactors that are arranged in series and the reaction product exiting the first reactor is subjected to chromatographic separation before it enters the second reactor. Preferably, the chromatographic separation is integrated in a simulated moving bed.

Allulose (psicose) is a low calorie sugar with the similar clean, sweet taste of sugar. Allulose is one of many different sugars that exists in nature in very small quantities. It was initially identified from wheat and has since been found in certain fruits including jackfruit, figs and raisins. Allulose is naturally present in small quantities in a variety of sweet foods like caramel sauce, maple syrup and brown sugar. Allulose is absorbed by the body, but not metabolized so it is nearly calorie-free.

H. Itoh et al., Journal of Fermentation and Bioengeneering, 80(1), 1995, 101-103 discloses preparation of D-psicose from D-fructose by immobilized D-tagatose 3-epimerase.

N. Wagner et al., Org. Process Res. Dev. 2012, 16, 323-330 relates to practical aspects of integrated operation of biotransformation and simulated moving bed (SMB) separation for fine chemical synthesis. D-psicose is produced using D-tagatose epimerase-catalyzed epimerization from D-fructose.

N. Wagner et al., Chemical Engineering Science 137 (2015) 423-435 relates to model-based cost optimization of a reaction-separation integrated process for the enzymatic production of the rare sugar D-psicose at elevated temperatures.

N. Wagner et al., Angew Chem Int Ed Engl. 2015, 54(14), 4182-6 discloses a separation-integrated cascade reaction to overcome thermodynamic limitations in rare-sugar synthesis.

A. Bosshart et al., Biotechnol Bioeng. 2016, 113(2), 349-58 relates to production of rare sugars D-psicose and L-tagatose by two engineered D-tagatose epimerases.

N. Wagner, et al., Journal of Chromatography A 2015, 1398, 47-56 relates to multi-objective optimization for the economic production of d-psicose using simulated moving bed chromatography.

The processes for the provision of product saccharides, preferably allulose, from educt saccharides, preferably from fructose, according to the prior art are not satisfactory in every respect and there is a demand for improvements.

It is an object of the invention to provide a process for the provision of product saccharides, preferably of allulose having advantages compared to the prior art.

This object has been achieved by the subject-matter of the patent claims.

It has been surprisingly found that by operating with more than one reactor among the SMB-separation the conversion of an educt saccharide, preferably of fructose to a product saccharide, preferably to allulose is strongly enhanced in comparison to its chemical equilibrium in batch setup. Thus, the space-time yield increases and the required amount of eluent is reduced. Apparently, the plant requires less recycle streams and conditioning and so gets more energy efficient than comparable processes like those presented by N Wagner et al.

The simulated moving bed (SMB) process is a highly engineered process for implementing chromatographic separation. The improved economic performance of SMB is brought about by a valve-and-column arrangement that is used to lengthen the stationary phase indefinitely and allow very high solute loadings to the process. In the simulated moving bed technique instead of moving the bed, the feed inlet, the solvent or eluent inlet and the desired product exit and undesired product exit positions are moved continuously, giving the impression of a moving bed, with continuous flow of solid particles and continuous flow of liquid in the opposite direction of the solid particles. True moving bed chromatography is only a theoretical concept. Its simulation is achieved by the use of a multiplicity of columns in series and a complex valve arrangement, which provides for flow of the feed mixture and solvent, and “eluent” or “desorbent” feed at any column. The valve and piping arrangements and the predetermined control of these allow switching at regular intervals the sample entry in one direction, the solvent entry in the same direction but at a different location in the continuous loop, whilst changing the fast product and slow product takeoff positions to also move in the same direction, but at different relative locations within the loop.

The process according to the invention preferably involves the following steps:
(a) providing a starting material comprising educt saccharide, preferably fructose;
(b) optionally, mixing the starting material with water or with an aqueous liquid and adjusting the concentration of dissolved educt saccharide, preferably fructose thereby providing a starting composition;
(c) converting educt saccharide, preferably fructose to product saccharide, preferably allulose under heterogeneous or homogeneous catalysis which includes chemical and/or enzymatic catalysis thereby providing a crude product composition; optionally providing said educt saccharide, preferably fructose from a precursor saccharide, preferably glucose using a second catalyst, which is either chemical or enzymatic, preferably in the same reactor;
(d) optionally, pre-purifying the crude product composition thereby providing a pre-purified product composition;
(e) optionally, concentrating the crude or the pre-purified product composition thereby providing a concentrated product composition;
(f) optionally, purifying the concentrated product composition by chromatography thereby providing a purified product saccharide composition;
(g) optionally, concentrating the purified product saccharide composition thereby providing a concentrated product saccharide composition;
(h) providing a liquid product saccharide product or a solid product saccharide product;
(i′) optionally, drying the solid product saccharide product thereby providing a dried product saccharide product;
(j) optionally, packaging the liquid product saccharide product or the dried product saccharide product thereby providing a packaged product saccharide product;
(k) optionally, palletizing the packaged product saccharide product thereby providing a palletized product saccharide product; and
(l) optionally, storing the packaged product saccharide product or the palletized product saccharide product.

The conversions of the educt saccharide to the product saccharide according to step (c) are preferably performed under enzymatic catalysis. The enzyme of choice depends upon the nature of the educt saccharide and on the nature of the product saccharide. Suitable enzymes for catalysis of a given conversion are known to a skilled person and commercially available. Preferred enzymes include phosphorylases and isomerases (e.g. epimerases).

In a preferred embodiment, the educt saccharide is a monosaccharide, preferably fructose, the product saccharide is a monosaccharide, preferably allulose, and the conversions according to step (c) are performed under enzymatic catalysis preferably by D-tagatose 3-epimerase.

In another preferred embodiment, the educt saccharide is a monosaccharide, preferably glucose, the product saccharide is a monosaccharide, preferably fructose, and the conversions according to step (c) are performed under enzymatic catalysis by glucose-fructose-epimerase.

In still another preferred embodiment, the educt saccharide is a monosaccharide, preferably fructose, the product saccharide is a monosaccharide, preferably tagatose, and the conversions according to step (c) are performed under enzymatic catalysis by tagatose-3-epimerase.

In yet another preferred embodiment, the educt saccharide is a monosaccharide, preferably galactose, the product saccharide is a monosaccharide, preferably tagatose, and the conversions according to step (c) are performed under enzymatic catalysis by tagatose-3-epimerase.

In a further preferred embodiment, the educt saccharide is a mixture of two monosaccharides, preferably in approximately equimolar ratio, preferably glucose-1-phosphate and glucose, the product saccharide is a disaccharide, preferably cellobiose, and the conversions according to step (c) are performed under enzymatic catalysis by cellobiose phosphorylase.

In another preferred embodiment, the educt saccharide is a mixture of two monosaccharides, preferably in approximately equimolar ratio, preferably glucose-1-phosphate and fructose, the product saccharide is a disaccharide, preferably sucrose, and the conversions according to step (c) are performed under enzymatic catalysis by sucrose phosphorylase.

Steps (a), (c), and (h) of the process according to the invention are mandatory, whereas steps (b), (e), (f), (g), (i′), (j), (k) and (l) are optional. Some of the optional steps depend upon one another.

For example, storing the packaged product saccharide product in step (l) requires the preceding packaging of the liquid product saccharide product or the dried product saccharide product in step (j). Likewise, storing the palletized product saccharide product in step (l) requires the preceding palletizing of the packaged product saccharide product in step (k) as well as the preceding packaging of the liquid product saccharide product or the dried product saccharide product in step (j).

Furthermore, depending upon the method of enzymatic conversion, some steps may be coupled with one another. For example, enzymatic conversion in a membrane reactor according to step (c) is preferably coupled with ultrafiltration according to step (d) (corresponding to a subsequence of steps -(c)-(d)-). Likewise, enzymatic conversion in a chromatographic reactor or an immobilized column reactor (Hashimoto process) according to step (c) is preferably coupled with chromatographic purification according to step (f) (corresponding to a subsequence of steps -(c)-(f)-, preferably omitting steps (d) and (e)).

Preferably, the steps are performed in alphabetical order. Consecutive steps may be timely separated from one another, i.e. the subsequent step may commence after the preceding step has been terminated, or at least partially simultaneously.

In preferred embodiments, the process according to the invention comprises or essentially consists of steps (a)-(c)-(h); (a)-(b)-(c)-(h); (a)-(c)-(d)-(h); (a)-(c)-(e)-(h); (a)-(c)-(f)-(h); (a)-(c)-(g)-(h); (a)-(c)-(h)-(i′); (a)-(c)-(d)-(e)-(h); (a)-(c)-(d)-(f)-(h); (a)-(c)-(d)-(g)-(h); (a)-(c)-(e)-(f)-(h); (a)-(c)-(e)-(g)-(h); (a)-(c)-(f)-(g)-(h); (a)-(c)-(d)-(e)-(f)-(h); (a)-(c)-(d)-(e)-(g)-(h); (a)-(c)-(d)-(f)-(g)-(h); (a)-(c)-(e)-(f)-(g)-(h); (a)-(c)-(e)-(f)-(g)-(h); (a)-(c)-(d)-(e)-(h)-(i′); (a)-(c)-(d)-(f)-(h)-(i′); (a)-(c)-(d)-(g)-(h)-(i′); (a)-(c)-(e)-(f)-(h)-(i′); (a)-(c)-(e)-(g)-(h)-(i′); (a)-(c)-(f)-(g)-(h)-(i′); (a)-(c)-(d)-(e)-(f)-(h)-(i′); (a)-(c)-(d)-(e)-(g)-(h)-(i′); (a)-(c)-(d)-(f)-(g)-(h)-(i′); (a)-(c)-(e)-(f)-(g)-(h)-(i′); (a)-(c)-(e)-(f)-(g)-(h)-(i′); (a)-(b)-(c)-(h)-(i′); (a)-(c)-(d)-(h)-(i′); (a)-(c)-(e)-(h)-(i′); (a)-(c)-(f)-(h)-(i′); (a)-(c)-(g)-(h)-(i′); (a)-(c)-(h)-(i′); (a)-(b)-(c)-(d)-(h); (a)-(b)-(c)-(e)-(h); (a)-(b)-(c)-(f)-(h); (a)-(b)-(c)-(g)-(h); (a)-(b)-(c)-(d)-(h)-(i′); (a)-(b)-(c)-(e)-(h)-(i′); (a)-(b)-(c)-(f)-(h)-(i′); (a)-(b)-(c)-(g)-(h)-(i′); (a)-(b)-(c)-(d)-(e)-(h); (a)-(b)-(c)-(d)-(f)-(h); (a)-(b)-(c)-(d)-(g)-(h); (a)-(b)-(c)-(e)-(f)-(h); (a)-(b)-(c)-(e)-(g)-(h); (a)-(b)-(c)-(f)-(g)-(h); (a)-(b)-(c)-(d)-(e)-(f)-(h); (a)-(b)-(c)-(d)-(e)-(g)-(h); (a)-(b)-(c)-(d)-(f)-(g)-(h); (a)-(b)-(c)-(e)-(f)-(g)-(h); (a)-(b)-(c)-(d)-(e)-(h)-(i′); (a)-(b)-(c)-(d)-(f)-(h)-(i′); (a)-(c)-(d)-(g)-(h)-(i′); (a)-(c)-(e)-(f)-(h)-(i′); (a)-(b)-(c)-(e)-(g)-(h)-(i′); (a)-(b)-(c)-(f)-(g)-(h)-(i′); (a)-(c)-(d)-(e)-(f)-(h)-(i′); (a)-(b)-(c)-(d)-(e)-(g)-(h)-(i′); (a)-(c)-(d)-(f)-(g)-(h)-(i′); (a)-(b)-(c)-(e)-(f)-(g)-(h)-(i′); or (a)-(b)-(c)-(e)-(f)-(g)-(h)-(i′).

In mandatory step (a) of the process according to the invention, a starting material comprising educt saccharide, preferably fructose is provided.

Alternatively, the educt saccharide may be a mixture of two different saccharides, e.g. glucose-1-phosphate and glucose, that are to be converted to cellobiose as product saccharide.

For the purpose of the specification, educt saccharide, preferably fructose refers to D-educt saccharide, preferably D-fructose which principally may also comprise minor amounts of L-educt saccharide, preferably L-fructose. Preferably, the educt saccharide, preferably fructose essentially is pure D-educt saccharide, preferably D-fructose, i.e. preferably does not comprise L-educt saccharide, preferably L-fructose.

The educt saccharide, preferably fructose may principally be provided in any form, e.g. as a solid, preferably crystalline material, or as a liquid, e.g. as an aqueous syrup.

The educt saccharide, preferably fructose may be provided in purified form or in admixture with other carbohydrates, especially monosaccharides and/or disaccharides, such as precursor saccharide, preferably glucose or sucrose.

In a preferred embodiment, the educt saccharide, preferably fructose is provided in form of a precursor saccharide/educt saccharide syrup, preferably glucose/fructose syrup, preferably based on sugar beet, sugar cane, maize (corn), wheat, tapioca, rice, palm, palm fruit, agave, maple, honey or jerusalem artichoke.

In another preferred embodiment, the educt saccharide, preferably fructose is provided in form of a precursor saccharide/educt saccharide syrup, preferably glucose/fructose syrup, preferably as described above, wherein the precursor saccharide, preferably glucose has been subsequently isomerized to educt saccharide, preferably fructose such that the residual content of precursor saccharide, preferably glucose has been reduced compared to the original content. Suitable methods for isomerization of precursor saccharide, preferably glucose to educt saccharide, preferably fructose thereby enriching the educt saccharide, preferably fructose content are known to a skilled person. For example, glucose can be isomerized to fructose using either Lewis acid or Brønsted base catalysts. Alternatively, glucose can be isomerized to fructose using fructose-glucose-isomerase for enzymatic catalysis.

In a preferred embodiment, a second enzyme is used for the preceding conversion of precursor saccharide, preferably glucose into educt saccharide, preferably fructose which performs parallel with the enzyme which subsequently converts the thus provided educt saccharide, preferably fructose to product saccharide, preferably allulose. Preferably, both enzymes are present in the same reactor so that less equipment is needed and the overall efficiency of the process is improved. The precursor saccharide, preferably glucose may originate from sucrose that in turn has been converted into e.g. invert sugar, i.e. an equimolar mixture of precursor saccharide, preferably glucose and educt saccharide, preferably fructose. Thus, the staring material may be a mixture of a precursor saccharide portion, preferably glucose portion and an educt saccharide portion, preferably fructose portion (preferably originating from sucrose) and the precursor saccharide portion, preferably glucose portion may be enzymatically converted into another educt saccharide portion, preferably fructose portion. Both educt saccharide portions, preferably fructose portions may then subsequently be converted to product saccharide, preferably allulose, preferably in one reactor.

In a preferred embodiment the conversion from educt saccharide, preferably fructose to product saccharide, preferably allulose takes place under heterogeneous or homogeneous catalysis, i.e. in the presence of a heterogeneous or homogeneous catalyst.

In still another preferred embodiment, the educt saccharide, preferably fructose is provided in form of a co-product provided by another process. For example, WO 2016/038142, which is incorporated by reference, discloses a process for the preparation of a product glucoside, preferably cellobiose, and of a co-product, preferably fructose, from an educt glucoside, preferably sucrose, with enzymatic catalysis. The educt glucoside is thereby first cleaved enzymatically to glucose 1-phosphate and the co-product, preferably fructose, and the glucose 1-phosphate is subsequently reacted to give the product glucoside. The co-product formed in the cleavage of the educt glucoside, preferably fructose, and the product glucoside formed in the reaction of the glucose 1-phosphate, are preferably each isolated. Thus, according to said another preferred embodiment of the invention, the educt saccharide, preferably fructose, which has been provided as co-product in the process according to e.g. WO2016038142, may be provided as starting material in step (a) of the process according to the invention.

In optional step (b) of the process according to the invention, the starting material provided in step (a) is mixed with water or with an aqueous liquid and the concentration of dissolved educt saccharide, preferably fructose is adjusted thereby providing a starting composition. Thus, the starting composition is an aqueous liquid.

When the starting material provided in step (a) is a solid material, e.g. crystalline educt saccharide, preferably crystalline fructose, in step (b) of the process according to the invention the solid material is preferably dissolved in water (e.g. tap water, or demineralized water or distilled water) or in an aqueous liquid which may already contain other constituents that are helpful for further processing such as buffers, electrolytes, cofactors, and the like. Suitable electrolytes include but are not limited to sodium, potassium, cobalt, manganese, phosphate, and the like. A preferred concentration of Mn²⁺ or Mg²⁺ is 1 mM. A suitable buffer is TRIS/HCl, e.g. at a concentration of 50 mM, for e.g. pH 7.5, or pH 9.0. However, buffers are not absolutely required in order to adjust and maintain a given pH value. Alternatively, the pH value can also be adjusted and maintained by titration with the necessary amount of a strong base, e.g. potassium hydroxide or sodium hydroxide.

When the starting material provided in step (a) is a liquid material, e.g. educt saccharide syrup, preferably fructose syrup, the educt saccharide, preferably fructose typically is already dissolved but at a concentration that is too high for further processing. Thus, under these circumstances, in step (b) of the process according to the invention the liquid material is preferably diluted with water or with an aqueous liquid which may already contain other constituents that are helpful for further processing.

In either case, the water or the aqueous liquid may originate from the process itself. In a preferred embodiment, the water or the aqueous liquid comprises a condensate or a side stream that has been provided in a subsequent concentration step and/or drying step of the process according to the invention, preferably in any of steps (e), (g) and/or (i′) of the process according to the invention.

In either case, the concentration of the educt saccharide, preferably fructose in the thus provided starting composition is adjusted to the desired concentration. Preferably, the concentration of the educt saccharide, preferably fructose is adjusted to a concentration within the range of from 5.0 wt.-% to 80 wt.-%, more preferably 5.0 wt.-% to 70 wt.-%, still more preferably from 20 wt.-% to 60 wt.-%, based on the total weight of the starting composition. In preferred embodiments, said concentration is within the range of 20±10 wt.-%, or 25±10 wt.-%, or 30±10 wt.-%, or 35±10 wt.-%, or 40±10 wt.-%, or 45±10 wt.-%, or 50±10 wt.-%, or 55±10 wt.-%, 60±10 wt.-%, or 70±10 wt.-%, or 80±10 wt.-%.

The pH value of the starting composition may be adjusted by addition of acids, bases or suitable buffer systems. Preferably, the pH value of the starting composition is within the range of from pH 2 to pH 12, preferably from pH 3 to pH 11. In preferred embodiments, said pH value is within the range of pH 3.0±1.0, or pH 3.5±1.0, or pH 4.0±1.0, or pH 4.5±1.0, or pH 5.0±1.0, or pH 5.5±1.0, or pH 6.0±1.0, or pH 6.5±1.0, or pH 7.0±1.0, or pH 7.5±1.0, or pH 8.0±1.0, or pH 8.5±1.0, or pH 9.0±1.0, or pH 9.5±1.0, or pH 10.0±1.0.

Before the starting composition is subjected to subsequent step (c) it may be filtered in order to remove undissolved residual material, e.g. by means of a filter having an average pore size of 0.2 m.

In mandatory step (c) of the process according to the invention, the educt saccharide, preferably fructose is converted (epimerized) to product saccharide, preferably allulose, preferably under enzymatic catalysis, thereby providing a crude product composition. Preferably, the crude product composition is an aqueous liquid.

For the purpose of the specification, product saccharide, preferably allulose (psicose) refers to D-product saccharide, preferably D-allulose which principally may also comprise minor amounts of L-product saccharide, preferably L-allulose. Preferably, the product saccharide, preferably allulose essentially is pure D-product saccharide, preferably D-allulose, i.e. preferably does not comprise L-product saccharide, preferably L-allulose.

The process according to the invention is preferably an enzymatic process, that is to say it takes place with enzymatic catalysis. The enzyme of choice depends upon the nature of the educt saccharide and on the nature of the product saccharide. Suitable enzymes for catalysis of a given conversion are known to a skilled person and commercially available. Preferred enzymes include phosphorylases and isomerases (e.g. epimerases).

The enzyme for the enzymatic conversion of fructose to e.g. allulose or tagatose should be a fructose-allulose-epimerase or fructose-tagatose-epimerase. Suitable methods for isomerization of precursor saccharide, preferably glucose to educt saccharide, preferably fructose thereby enriching the educt saccharide, preferably fructose content are known to a skilled person. According to a preferred embodiment, the fructose-allulose-epimerase could be a D-tagatose 3-epimerase (EC 5.1.3.31), e.g. from Pseudomonas cichorii, is a preferred enzyme which may be expressed in host organisms such as Bacillus spp., Pichia spp. or E. coli, preferably E. coli JM109 or other K12 derivates or E. coli BL21 or other B derivates.

Preferably, the D-tagatose 3-epimerase is from a bacterium selected from the group consisting of Pseudomonas sp., Rhodobacter sp. and Mesorhizobium sp. The enzymes from the bacteria Pseudomonas cichorii, Pseudomonas sp. ST-24, Rhodobacter sphaeroides and Mesorhizobium loti are all suitable as they catalyze the epimerization of various ketoses at the C₃ position, interconverting D-fructose and D-psicose, D-tagatose and D-sorbose, D-ribulose and D-xylulose, and L-ribulose and L-xylulose. The specificity depends on the species. The enzymes from Pseudomonas cichorii and Rhodobacter sphaeroides may require a co-factor such as Mn²⁺ or Mg²⁺.

It has been surprisingly found that the D-tagatose 3-epimerase and additional enzymes, if any, may be employed repeatedly (i.e. recycled), for example by carrying out step (c) in one or more membrane reactors, and that said enzymes do not need to be immobilized at solid supports that are located in separate reaction vessels (reactors). Further, there is no requirement for inactivating said enzymes after the reaction.

Preferably, step (c) is carried out in a single aqueous phase which essentially contains no organic solvents.

The enzyme may be employed in isolated, purified form or in form of the crude extract (cell free, lyophilized fermentation broth).

The enzyme may be freely dissolved or immobilized on a solid carrier. The enzyme may be present in dissolved state, i.e. free in solution, and may be retained in the reactor by membranes. Alternatively, the enzyme may be immobilized on a solid support. Alternatively, the enzyme may be present in microorganisms that in turn are retained in the reactor by membranes. Alternatively, the enzyme may be present in microorganisms that in turn are immobilized on a solid support.

When the enzymes or the microorganisms containing the enzymes are immobilized on a solid support, the solid support material is not particularly limited and may include resins, plastics, glass, and the like. The enzyme may also be encapsulated by the solid support material, e.g. in form of alginate beads). When microorganisms containing the enzymes are immobilized, they may be free or densely packed in the reactor.

Conversion temperatures are preferably within the range of from 10° C. to 90° C., more preferably from 20° C. to 70° C. Preferably, the enzymatic conversion is performed at a temperature within the range of from 20° C. to 60° C., more preferably 20° C. to 60° C. The ideal reaction temperature depends upon the activity and temperature stability of the enzyme and may be determined by routine testing. In preferred embodiments, the temperature is within the range of 10±5° C., or 15±5° C., or 20±5° C., or 25±5° C., or 30±5° C., or 35±5° C., or 40±5° C., 45±5° C. or 50±5° C. or 55±5° C. or 60±5° C. or 65±5° C. or 70±5° C. or 75±5° C. or 80±5° C.

Appropriate electrolytes may be present, such as sodium, potassium, cobalt, manganese, magnesium, phosphate, and the like, or the conversion may be performed essentially in the absence of electrolytes.

When the enzymatic conversion is performed with freely dissolved or with immobilized enzyme, the conversion may be performed continuously or batch-wise. Further substrate (i.e. starting material, educt saccharide, preferably fructose) may be added by fedbatch during the conversion.

When the reaction is performed batch-wise, typical reaction times may be within the range of from several minutes to several days, e.g. about 30 minutes to 36 hours.

If desirable, the product saccharide, preferably allulose may not be isolated, but may be used as an intermediate for further synthesis. For example, product saccharide, preferably allulose may be converted in situ to allose by means of a second enzyme, which in turn may also independently be freely dissolved or immobilized (Y. R. Lim et al., Appl Microbiol Biotechnol 2011, 91(2), 229-35).

In a preferred embodiment, the educt saccharide, preferably fructose is converted to product saccharide, preferably allulose according to a so-called Hashimoto process involving chromatographic reactors, preferably immobilized column reactors, combining biochemical conversion with chromatographic separation. This is typically achieved by coupling a flow reactor unit with immobilized enzyme therein with a subsequent chromatographic unit such that educt saccharide, preferably fructose in the reaction mixture, while flowing through the reactor unit, is converted to product saccharide, preferably allulose and subsequently enters the chromatography unit for separation of product saccharide, preferably allulose and residual (i.e. non-converted) educt saccharide, preferably fructose. Under these circumstances, the subsequent purifying by chromatography in step (f) is integrated in the enzymatic conversion in step (c).

Preferably, this aspect of the invention relates to process for the synthesis of product saccharide, preferably allulose in at least two reactors R₁ and R₂, the method comprising the steps of
(i) supplying a liquid comprising educt saccharide, preferably fructose, typically an aqueous solution of educt saccharide, preferably fructose, to the reactor R₁ and converting a portion of the educt saccharide, preferably fructose to product saccharide, preferably allulose under enzymatic catalysis thereby providing a liquid comprising product saccharide, preferably allulose and residual educt saccharide, preferably fructose;
(ii) separating at least a portion of the product saccharide, preferably allulose from the residual educt saccharide, preferably fructose of step (i) by liquid chromatography thereby providing
   a first chromatographic fraction comprising residual educt saccharide, preferably fructose and optionally product saccharide, preferably allulose; and
   a second chromatographic fraction comprising product saccharide, preferably allulose and optionally residual educt saccharide, preferably fructose; and
(iii) supplying the first chromatographic fraction of step (ii) to the reactor R₂ and converting at least a portion of the residual educt saccharide, preferably fructose to product saccharide, preferably allulose under enzymatic catalysis.

According to a preferred variant of the process according to the invention, the reactors R₁ and R₂ both contain two enzymes,
an enzyme capable of catalyzing the conversion of the precursor saccharide to the educt saccharide (e.g. an educt saccharide-precursor saccharide-isomerase, preferably glucose-fructose-isomerase), as well as
an enzyme capable of catalyzing the conversion of the educt saccharide to the product saccharide (e.g. a product saccharide-educt saccharide-isomerase, preferably allulose-fructose-epimerase).

According to this preferred variant, the liquid supplied in step (i) comprises precursor saccharide, preferably glucose, which is optionally present in admixture with educt saccharide, preferably fructose (e.g. invert sugar). The liquid comprising precursor saccharide, preferably glucose is supplied to the reactor R₁ where a portion of the precursor saccharide, preferably glucose is converted to educt saccharide, preferably fructose under enzymatic catalysis (enzyme capable of catalyzing the conversion of the precursor saccharide to the educt saccharide, e.g. an educt saccharide-precursor saccharide-isomerase, preferably glucose-fructose-isomerase) thereby providing a liquid comprising educt saccharide, preferably fructose and residual precursor saccharide, preferably glucose; simultaneously, a portion of the educt saccharide, preferably fructose is converted to product saccharide, preferably allulose under enzymatic catalysis (enzyme capable of catalyzing the conversion of the educt saccharide to the product saccharide, e.g. a product saccharide-educt saccharide-isomerase, preferably allulose-fructose-epimerase) thereby providing a liquid comprising product saccharide, preferably allulose and residual educt saccharide, preferably fructose and residual precursor saccharide, preferably glucose.

According to this preferred variant, subsequent separation in step (ii) by liquid chromatography provides a first chromatographic fraction comprising residual educt saccharide, preferably fructose and optionally product saccharide, preferably allulose and optionally residual precursor saccharide, preferably glucose; and a second chromatographic fraction comprising product saccharide, preferably allulose and optionally residual educt saccharide, preferably fructose and optionally residual precursor saccharide, preferably glucose; and a further chromatographic fraction comprising precursor saccharide, preferably glucose and optionally residual educt saccharide, preferably fructose and optionally product saccharide, preferably allulose.

According to this preferred variant, in step (iii) the first chromatographic fraction as well as the further chromatographic fraction of step (ii) are supplied to the reactor R₂ and
at least a portion of the residual educt saccharide, preferably fructose is converted to product saccharide, preferably allulose under enzymatic catalysis (enzyme capable of catalyzing the conversion of the educt saccharide to the product saccharide, e.g. a product saccharide-educt saccharide-isomerase, preferably allulose-fructose-epimerase) and
at least a portion of the residual precursor saccharide, preferably glucose is converted to educt saccharide, preferably fructose under enzymatic catalysis (enzyme capable of catalyzing the conversion of the precursor saccharide to the educt saccharide, e.g. an educt saccharide-precursor saccharide-isomerase, preferably glucose-fructose-isomerase).

In a preferred embodiment of the process according to the invention, the conversion of educt saccharide, preferably fructose to product saccharide, preferably allulose according to step (i) and/or step (iii) is performed under enzymatic catalysis, preferably by a single enzyme.

In another preferred embodiment of the process according to the invention, the conversion of educt saccharide, preferably fructose to product saccharide, preferably allulose according to step (i) and/or step (iii) is performed under chemical heterogeneous or homogeneous catalysis.

In still another preferred embodiment of the process according to the invention, precursor saccharide, preferably glucose is converted to educt saccharide, preferably fructose under enzymatic catalysis in the same reactor parallel to the conversion of educt saccharide, preferably fructose to product saccharide, preferably allulose according to step (i) and/or step (iii). Thus, at least a portion of the precursor saccharide, preferably glucose is converted to educt saccharide, preferably fructose and at least a portion of the thus obtained educt saccharide, preferably fructose which in turn is converted to product saccharide, preferably allulose.

In yet another preferred embodiment of the process according to the invention, precursor saccharide, preferably glucose is converted to educt saccharide, preferably fructose under chemical heterogeneous or homogeneous catalysis in the same reactor parallel to the conversion of educt saccharide, preferably fructose to product saccharide, preferably allulose according to step (i) and/or step (iii). Thus, at least a portion of the precursor saccharide, preferably glucose is converted to educt saccharide, preferably fructose and at least a portion of the thus obtained educt saccharide, preferably fructose which in turn is converted to product saccharide, preferably allulose.

Steps (i) to (iii) as described above may then be integrated in the process comprising at least steps (a) and (c) as described above, wherein steps (i) to (iii) replace steps (c) and optional steps (d), (e) and (f). Thus, when integrating both processes into one another, the resultant process according to the invention preferably involves the following steps:
(a) providing a starting material comprising educt saccharide, preferably fructose;
(b) optionally, mixing the starting material with water or with an aqueous liquid and adjusting the concentration of dissolved educt saccharide, preferably fructose thereby providing a starting composition;
(i) supplying a liquid comprising educt saccharide, preferably fructose or the starting composition to the reactor R₁ and converting a portion of the educt saccharide, preferably fructose to product saccharide, preferably allulose under enzymatic catalysis thereby providing a liquid comprising product saccharide, preferably allulose and residual educt saccharide, preferably fructose;
(ii) separating at least a portion of the product saccharide, preferably allulose from the residual educt saccharide, preferably fructose of step (i) by liquid chromatography thereby providing
   a first chromatographic fraction comprising residual educt saccharide, preferably fructose and optionally product saccharide, preferably allulose; and
   a second chromatographic fraction comprising product saccharide, preferably allulose and optionally residual educt saccharide, preferably fructose;
(iii) supplying the first chromatographic fraction of step (ii) to the reactor R₂; converting at least a portion of the residual educt saccharide, preferably fructose to product saccharide, preferably allulose under enzymatic catalysis; and providing a purified product saccharide composition;
(g) optionally, concentrating the purified product saccharide composition thereby providing a concentrated product saccharide composition;
(h) providing a liquid product saccharide product or a solid product saccharide product;
(i′) optionally, drying the solid product saccharide product thereby providing a dried product saccharide product;
(j) optionally, packaging the liquid product saccharide product or the dried product saccharide product thereby providing a packaged product saccharide product;
(k) optionally, palletizing the packaged product saccharide product thereby providing a palletized product saccharide product; and
(l) optionally, storing the packaged product saccharide product or the palletized product saccharide product.

Supplying step (i) is not to be confused with optional drying step (i′).

One or more of the optional process steps (iv) to (xi) described in detail hereinafter may also be performed, preferably after step (iii) and before step (g).

Chromatographic reactors and immobilized column reactors in general provide an option of combining chemical and biochemical reactions, respectively, with chromatographic separation thereby integrating several process steps in one and the same facility. Especially by means of counterflow processes such as simulated moving bed (SMB) chromatography, equilibria may be overcome thereby achieving substantial improvements of productivity. The simulated moving bed (SMB) chromatography is achieved by the use of a multiplicity of columns in series and a complex valve arrangement, which provides for sample and solvent feed, and also product and non-reacted educt takeoff at appropriate locations of any column, whereby it allows switching at regular intervals the sample entry in one direction, the solvent entry in the opposite direction, whilst changing the product and non-reacted educt takeoff positions appropriately as well.

The integration of chemical reactions into chromatographic separations offers the potential to improve the conversion of equilibrium-limited reactions. By the simultaneous removal of the products, the reaction equilibrium is shifted to the side of the products. This combination of reaction and chromatographic separation can be achieved by packing the columns of the SMB process uniformly with adsorbent and catalyst, which leads to the reactive (SMBR) process.

The SMBR process can be advantageous in terms of higher productivity in comparison to a sequential arrangement of reaction and separation units. However, a uniform catalyst distribution in the SMBR promotes the backward reaction near the product outlet which is detrimental for the productivity. The renewal of deactivated catalyst is difficult when it is mixed with adsorbent beads, and the same conditions must be chosen for separation and reaction which may lead to either suboptimal reaction or suboptimal separation.

The Hashimoto SMB process overcomes the disadvantages of the SMBR by performing separation and reaction in separate units that contain only adsorbent or only catalyst. In this configuration, the conditions for reaction and for separation can be chosen separately, and the reactors can constantly be placed in the separation zones of the SMB process by appropriate switching.

The Hashimoto process is based upon the SMB-principle and combines simulated moving bed chromatography in columns with synthesis in reactors. (T. Borren et al., Chemie Ingenieur Technik 2004, 76(9), 1291-2).

Depending upon the individual design, a Hashimoto process may comprise several zones. In the Hashimoto process the functionalities of separation and reaction are performed in different columns and the reactors are fixed in the separation zones. The practical realization of the port shifting and the fixed reactor positions relative to the Ports is demanding, since each reactor must be connected to each separative column once over the full cycle of operation.

The Hashimoto SMB process can be implemented as a three-zone process or as a four-zone process. Preferably, the Hashimoto SMB process is implemented as a four-zone process.

In the three-zone process, the feed stream is completely converted to a product stream with the required purity. The reactors and the separators are placed in alternating sequence in order to increase Conversion by reaching the reactive equilibrium within the reactor and removing the product in the following separating unit.

The four-zone process has an additional raffinate stream containing the educt (here educt saccharide, preferably fructose) and an additional zone IV in order to improve the regeneration of the eluent. Thereby, at the expense of an additional stream that is not the desired product and of additional columns, the process can be operated with smaller desorbent consumption or a higher feed throughput and a breakthrough of the components over the recycle stream can be prevented more easily (H. Schmidt-Traub et al., Integrated Reaction and Separation Operations: Modelling and experimental validation, Springer, 2006).

According to the present invention, zone III preferably comprises stationary reactors between the individual separation columns. Said reactors permanently remain within zone III and thus move along with pulsing of flow direction, thereby achieving a distinction of reaction and separation. Compared to homogenous mixture, such distinction has several advantages. For example, adsorbate and catalyst may be replaced and regenerated individually. Further, different optimized temperatures may be adjusted for separation on the one hand and for synthesis on the other hand in order to improve productivity.

Due to its high complexity, SMB chromatography requires rigorous modeling and simulations in order to dimension the facility and further to operate it. Also in this regard distinction between separation and synthesis is advantageous, as it does not require modeling of a column that otherwise would serve both purposes simultaneously, separation and synthesis.

When converting educt saccharide, preferably fructose to product saccharide, preferably allulose and separating product saccharide, preferably allulose from educt saccharide, preferably fructose according to the Hashimoto process, the required purity of the weaker adsorbing species present a limitation to the overall process. When the weaker adsorbing species is the product to be isolated (here product saccharide, preferably allulose), the number of stationary reactors may be increased in order to improve purity. The stronger adsorbing species can principally be isolated with high purity. Alternatively, a reactor upstream of the SMB facility can have advantages.

N. Wagner et al. use the combination of a reactor and SMB chromatography with recycle of non-reacted educt by a nanofiltration plant to the reactor in order to increase the apparent conversion. In comparison to that, the process according to the present invention operates with a multitude of reactors (more than one reactor) in order to shift the reaction equilibrium. The reactors are installed in the SMB setup in a consecutive manner, whereas preferably every reactor is followed by a chromatographic column such that reactors and chromatographic columns are arranged in an alternating manner. Once the reaction equilibrium is reached in the first reactor, the non-reacted educt (educt saccharide, preferably fructose) is separated from the product (product saccharide, preferably allulose) in the subsequent first chromatographic column. Said non-reacted educt (educt saccharide, preferably fructose) is supplied to a second reactor which is arranged after the first chromatographic column. Once the reaction equilibrium is reached in the second reactor, the non-reacted educt (educt saccharide, preferably fructose) is separated from the product (product saccharide, preferably allulose) in a subsequent second chromatographic column, and so on.

Thus, compared to the setup according to N. Wagner et al., the conversion in one passage according to the present invention is substantially higher at lower energy consumption.

The additional nanofiltration, which according to the setup of N. Wagner et al. is needed in order to improve the overall conversion, is not needed according to the present invention. Without such nanofiltration, the overall efficiency according to the present invention is higher. By placing the reactors in the SMB setup according to the present invention, the dilution factor due to the needed eluent is the same as in a normal SMB setup.

A first aspect of the invention in accordance with the Hashimoto process relates to process for the synthesis of product saccharide, preferably allulose in at least two reactors R₁ and R₂, the method comprising the steps of
(i) supplying a liquid comprising educt saccharide, preferably fructose to the reactor R₁ and converting a portion of the educt saccharide, preferably fructose to product saccharide, preferably allulose under enzymatic catalysis thereby providing a liquid comprising product saccharide, preferably allulose and residual educt saccharide, preferably fructose;
(ii) separating at least a portion of the product saccharide, preferably allulose from the residual educt saccharide, preferably fructose of step (i) by liquid chromatography thereby providing
   a first chromatographic fraction comprising residual educt saccharide, preferably fructose and optionally product saccharide, preferably allulose; and
   a second chromatographic fraction comprising product saccharide, preferably allulose and optionally residual educt saccharide, preferably fructose; and
(iii) supplying the first chromatographic fraction of step (ii) to the reactor R₂ and converting at least a portion of the residual educt saccharide, preferably fructose to product saccharide, preferably allulose under enzymatic catalysis.

According to a preferred variant of the process according to the invention, the reactors R₁ and R₂ both contain two enzymes,
an enzyme capable of catalyzing the conversion of the precursor saccharide to the educt saccharide (e.g. an educt saccharide-precursor saccharide-isomerase, preferably glucose-fructose-isomerase) as well as
an enzyme capable of catalyzing the conversion of the educt saccharide to the product saccharide (e.g. a product saccharide-educt saccharide-isomerase, preferably allulose-fructose-epimerase).

According to this preferred variant, the liquid supplied in step (i) comprises precursor saccharide, preferably glucose, which is optionally present in admixture with educt saccharide, preferably fructose (e.g. invert sugar). The liquid comprising precursor saccharide, preferably glucose is supplied to the reactor R₁ where a portion of the precursor saccharide, preferably glucose is converted to educt saccharide, preferably fructose under enzymatic catalysis (enzyme capable of catalyzing the conversion of the precursor saccharide to the educt saccharide, e.g. an educt saccharide-precursor saccharide-isomerase, preferably glucose-fructose-isomerase) thereby providing a liquid comprising educt saccharide, preferably fructose and residual precursor saccharide, preferably glucose; simultaneously, a portion of the educt saccharide, preferably fructose is converted to product saccharide, preferably allulose under enzymatic catalysis (enzyme capable of catalyzing the conversion of the educt saccharide to the product saccharide, e.g. a product saccharide-educt saccharide-isomerase, preferably allulose-fructose-epimerase) thereby providing a liquid comprising product saccharide, preferably allulose and residual educt saccharide, preferably fructose and residual precursor saccharide, preferably glucose.

According to this preferred variant, subsequent separation in step (ii) by liquid chromatography provides a first chromatographic fraction comprising residual educt saccharide, preferably fructose and optionally product saccharide, preferably allulose and optionally residual precursor saccharide, preferably glucose; and a second chromatographic fraction comprising product saccharide, preferably allulose and optionally residual educt saccharide, preferably fructose and optionally residual precursor saccharide, preferably glucose; and a further chromatographic fraction comprising precursor saccharide, preferably glucose and optionally residual educt saccharide, preferably fructose and optionally product saccharide, preferably allulose.

According to this preferred variant, in step (iii) the first chromatographic fraction as well as the further chromatographic fraction of step (ii) are supplied to the reactor R₂ and
at least a portion of the residual educt saccharide, preferably fructose is converted to product saccharide, preferably allulose (enzyme capable of catalyzing the conversion of the educt saccharide to the product saccharide, e.g. a product saccharide-educt saccharide-isomerase, preferably allulose-fructose-epimerase) and
at least a portion of the residual precursor saccharide, preferably glucose is converted to educt saccharide, preferably fructose under enzymatic catalysis (enzyme capable of catalyzing the conversion of the precursor saccharide to the educt saccharide, e.g. an educt saccharide-precursor saccharide-isomerase, preferably glucose-fructose-isomerase).

Typically, the relative weight ratio of residual educt saccharide, preferably fructose to product saccharide, preferably allulose in the first chromatographic fraction differs from the relative weight ratio of residual educt saccharide, preferably fructose to product saccharide, preferably allulose in the second chromatographic fraction, in each case relative to the total weight of product saccharide, preferably allulose and residual educt saccharide, preferably fructose in the first chromatographic fraction and in the second chromatographic fraction, respectively.

Preferably, the relative weight ratio of residual educt saccharide, preferably fructose to product saccharide, preferably allulose in the first chromatographic fraction is higher than the relative weight ratio of residual educt saccharide, preferably fructose to product saccharide, preferably allulose in the second chromatographic fraction, in each case relative to the total weight of product saccharide, preferably allulose and residual educt saccharide, preferably fructose in the first chromatographic fraction and in the second chromatographic fraction, respectively.

In preferred embodiments
the relative weight content of educt saccharide, preferably fructose in the first chromatographic fraction is at least 70 wt.-%, more preferably at least 75 wt.-%, still more preferably at least 80 wt.-%, yet more preferably at least 85 wt.-%, even more preferably at least 90 wt.-%, most preferably at least 95 wt.-%, and in particular at least 97.5 wt.-%, in each case relative to the total weight of the educt saccharide, preferably fructose and the product saccharide, preferably allulose in the first chromatographic fraction; and/or
the relative weight content of product saccharide, preferably allulose in the second chromatographic fraction is at least 70 wt.-%, more preferably at least 75 wt.-%, still more preferably at least 80 wt.-%, yet more preferably at least 85 wt.-%, even more preferably at least 90 wt.-%, most preferably at least 95 wt.-%, and in particular at least 97.5 wt.-%, in each case relative to the total weight of the educt saccharide, preferably fructose and the product saccharide, preferably allulose in the second chromatographic fraction.

Preferably, in step (ii) the residual educt saccharide, preferably fructose and optionally the residual precursor saccharide, preferably glucose has a shorter retention time than the product saccharide, preferably allulose.

Preferably, both chromatographic fractions are supplied to the reactor R₂, whereas the second chromatographic fraction is supplied to the reactor R₂ after the first chromatographic fraction.

Preferably, the conversion of step (iii) also provides product saccharide, preferably allulose and residual educt saccharide, preferably fructose and optionally residual precursor saccharide, preferably glucose.

In a preferred embodiment, the process according to the invention comprises the additional step of
(iv) separating at least a portion of the product saccharide, preferably allulose from the residual educt saccharide, preferably fructose of step (iii) by liquid chromatography thereby providing
   a third chromatographic fraction comprising residual educt saccharide, preferably fructose and optionally residual product saccharide, preferably allulose and optionally residual precursor saccharide, preferably glucose; and
   a fourth chromatographic fraction comprising product saccharide, preferably allulose and optionally residual educt saccharide, preferably fructose and optionally residual precursor saccharide, preferably glucose.

Preferably, the fourth chromatographic fraction is recirculated to step (i).

In preferred embodiments
the relative weight content of educt saccharide, preferably fructose in the third chromatographic fraction is at least 70 wt.-%, more preferably at least 75 wt.-%, still more preferably at least 80 wt.-%, yet more preferably at least 85 wt.-%, even more preferably at least 90 wt.-%, most preferably at least 95 wt.-%, and in particular at least 97.5 wt.-%, in each case relative to the total weight of the educt saccharide, preferably fructose and the product saccharide, preferably allulose in the third chromatographic fraction; and/or
the relative weight content of product saccharide, preferably allulose in the fourth chromatographic fraction is at least 70 wt.-%, more preferably at least 75 wt.-%, still more preferably at least 80 wt.-%, yet more preferably at least 85 wt.-%, even more preferably at least 90 wt.-%, most preferably at least 95 wt.-%, and in particular at least 97.5 wt.-%, in each case relative to the total weight of the educt saccharide, preferably fructose and the product saccharide, preferably allulose in the fourth chromatographic fraction.

According to the present invention, the series of reactor and liquid chromatography may involve more than the two reactors R₁ and R₂, i.e.
a reactor R₃ followed by a liquid chromatography for separating a fifth chromatographic fraction from a sixth chromatographic fraction;
a reactor R₄ followed by a liquid chromatography for separating a seventh chromatographic fraction from an eighth chromatographic fraction;
and so on.

For example, in a preferred embodiment, the process according to the invention comprises the additional steps of
(α) supplying the third chromatographic fraction of optional step (iv) to a reactor R₃ and converting at least a portion of the residual educt saccharide, preferably fructose to product saccharide, preferably allulose under enzymatic catalysis; and
(β) optionally, separating at least a portion of the product saccharide, preferably allulose from the residual educt saccharide, preferably fructose of step (a) by liquid chromatography thereby providing
   a fifth chromatographic fraction comprising residual educt saccharide, preferably fructose and optionally product saccharide, preferably allulose; and
   a sixth chromatographic fraction comprising product saccharide, preferably allulose and optionally residual educt saccharide, preferably fructose.

Preferably, however, the process according to the invention involves at most four such reactors, more preferably at most three such reactors, and most preferably the two reactors R₁ and R₂. Thus, while according to the invention being encompassed, in the following all preferred definitions are focused on two reactors and a skilled person recognizes that in case of three reactors or four reactors all definitions may apply in analogy also to the additional reactors and chromatography units, respectively.

Preferably, the liquid chromatography in step (ii) and/or in optional step (iv) is performed by means of an adsorbent bed comprising a calcium based resin. Preferably, the liquid chromatography in step (ii) and/or in optional step (iv) is performed at a temperature within the range of from 40° C. to 90° C.

Preferably, the conversions of educt saccharide, preferably fructose to product saccharide, preferably allulose according to step (i) and/or step (iii) are performed under enzymatic catalysis by a single enzyme. Preferably, when the educt saccharide is fructose and the product saccharide is allulose, the conversions according to step (i) and/or step (iii) are performed under enzymatic catalysis by D-tagatose 3-epimerase. Preferably, the D-tagatose 3-epimerase is from a bacterium selected from the group consisting of Pseudomonas sp., Rhodobacter sp. and Mesorhizobium sp. Preferably, the conversions according to step (i) and/or step (iii) are performed under enzymatic catalysis by an enzyme, wherein the enzyme
is present in dissolved state and is retained in the reactor R₁ and/or R₂ by membranes;
is immobilized on a solid support;
is present in microorganisms that in turn are retained in the reactor R₁ and/or R₂ by membranes; or
is present in microorganisms that are immobilized on a solid support.

More preferably, the conversions according to step (i) and/or step (iii) are performed under enzymatic catalysis by an enzyme, wherein the enzyme
is immobilized on a solid support; or
is present in microorganisms that are immobilized on a solid support.

Preferably, reactor R₁ and/or reactor R₂ is a membrane reactor or immobilized column reactor or a chromatographic reactor. More preferably, reactor R₁ and reactor R₂ are both chromatographic reactors or both immobilized column reactors. More preferably, reactor R₁ and reactor R₂ are both immobilized column reactors. For the purpose of the specification, unless expressly stated otherwise, a chromatographic reactor is a reactor in which enzyme is immobilized, optionally being incorporated in immobilized microorganisms, and which may be coupled to a subsequent adsorbent bed for chromatography. An immobilized column reactor is a subtype of such chromatographic reactor. The housing of reactor unit and chromatography unit is not particularly limited. Thus, reactor unit and chromatography unit may be contained in the same housing, e.g. column, or in separate housings.

For the purpose of the specification, the term “reactor” may refer to a single reactor or to a series of or cascade of individual reactors that are in flow connection with one another and may optionally be integrated in one and the same housing.

Preferably, the process according to the invention is performed continuously.

In a particularly preferred embodiment, the liquid chromatography of step (ii) and/or of optional step (iv) are integrated in a simulated moving bed (SMB).

Typically, liquid flows through the SMB in a flow direction and an adsorbent bed is simulated to move in opposite direction.

Preferably, the SMB comprises four zones I to IV, wherein liquid is cycled through zones I to IV and wherein with respect to flow direction of liquid zone IV is downstream zone III, zone III is downstream zone II, zone II is downstream zone I, and zone I is downstream zone IV.

Preferably, one of said four zones I to IV comprises in a downstream arrangement with respect to flow direction of liquid: the reactor R₁ for the conversion of step (i), a first adsorbent bed for the liquid chromatography of step (ii), the reactor R₂ for the conversion of step (iii), and optionally a second adsorbent bed for the liquid chromatography of optional step (iv).

Preferably, the SMB comprises
a zone I comprising one or more serial adsorbent beds C-Iₘ, wherein index m is an integer of at least 1, preferably at least 2 or at least 3;
a zone II comprising one or more serial adsorbent beds C-IIₙ, wherein index n is an integer of at least 1, preferably at least 2 or at least 3;
a zone III comprising the reactor R₁ for the conversion of step (i), the reactor R₂ for the conversion of step (iii), and one or more serial adsorbent beds C-IIIₚ, wherein index p is an integer of at least 1, preferably at least 2 or at least 3; wherein with respect to flow direction of liquid (eluent) at least one of said adsorbent beds C-IIIₚ is arranged downstream the reactor R₁ and upstream the reactor R₂; and
a zone IV comprising one or more serial adsorbent beds C-IV_{q}, wherein index p is an integer of at least 1, preferably at least 2 or at least 3.

Preferably, indices m, n, p and q are independently of one another within the range of from 1 to 12, more preferably in each case independently of one another 2, 3, 4, 5, 6, 7, 8, 9, 10, or 11.

Preferably, at least one of indices m, n, p and q is greater than 1.

In a preferred embodiment, indices m, n, p and q are identical. In another preferred embodiment, indices m, n, p and q are not identical, i.e. at least one integer differs from at least one other integer, whereas the remaining integers may also be different or identical with said at least integer or said at least one other integer.

In preferred embodiments A¹ to A¹⁵ of the process according to the invention, indices m, n, p and q have the following meaning:

| | A¹ | A² | A³ | A⁴ | A⁵ | A⁶ | A⁷ | A⁸ | A⁹ | A¹⁰ | A¹¹ | A¹² | A¹³ | A¹⁴ | A¹⁵ |
|---|---|---|---|---|---|---|---|---|---|---|---|---|---|---|---|
| m | 1 | 2 | 1 | 1 | 1 | 2 | 2 | 2 | 1 | 1 | 1 | 2 | 2 | 2 | 1 |
| n | 1 | 1 | 2 | 1 | 1 | 2 | 1 | 1 | 2 | 2 | 1 | 2 | 2 | 1 | 2 |
| p | 1 | 1 | 1 | 2 | 1 | 1 | 2 | 1 | 2 | 1 | 2 | 2 | 1 | 2 | 2 |
| q | 1 | 1 | 1 | 1 | 2 | 1 | 1 | 2 | 1 | 2 | 2 | 1 | 2 | 2 | 2 |

In preferred embodiments B¹ to B¹⁵ of the process according to the invention, indices m, n, p and q have the following meaning:

| | B¹ | B² | B³ | B⁴ | B⁵ | B⁶ | B⁷ | B⁸ | B⁹ | B¹⁰ | B¹¹ | B¹² | B¹³ | B¹⁴ | B¹⁵ |
|---|---|---|---|---|---|---|---|---|---|---|---|---|---|---|---|
| m | 2 | 3 | 2 | 2 | 2 | 3 | 3 | 3 | 2 | 2 | 2 | 3 | 3 | 3 | 2 |
| n | 2 | 2 | 3 | 2 | 2 | 3 | 2 | 2 | 3 | 3 | 2 | 3 | 3 | 2 | 3 |
| p | 2 | 2 | 2 | 3 | 2 | 2 | 3 | 2 | 3 | 2 | 3 | 3 | 2 | 3 | 3 |
| q | 2 | 2 | 2 | 2 | 3 | 2 | 2 | 3 | 2 | 3 | 3 | 2 | 3 | 3 | 3 |

In preferred embodiments C¹ to C¹⁵ of the process according to the invention, indices m, n, p and q have the following meaning:

| | C¹ | C² | C³ | C⁴ | C⁵ | C⁶ | C⁷ | C⁸ | C⁹ | C¹⁰ | C¹¹ | C¹² | C¹³ | C¹⁴ | C¹⁵ |
|---|---|---|---|---|---|---|---|---|---|---|---|---|---|---|---|
| m | 3 | 4 | 3 | 3 | 3 | 4 | 4 | 4 | 3 | 3 | 3 | 4 | 4 | 4 | 3 |
| n | 3 | 3 | 4 | 3 | 3 | 4 | 3 | 3 | 4 | 4 | 3 | 4 | 4 | 3 | 4 |
| p | 3 | 3 | 3 | 4 | 3 | 3 | 4 | 3 | 4 | 3 | 4 | 4 | 3 | 4 | 4 |
| q | 3 | 3 | 3 | 3 | 4 | 3 | 3 | 4 | 3 | 4 | 4 | 3 | 4 | 4 | 4 |

In preferred embodiments D¹ to D¹⁶ of the process according to the invention, indices m, n, p and q have the following meaning:

| | D¹ | D² | D³ | D⁴ | D⁵ | D⁶ | D⁷ | D⁸ | D⁹ | D¹⁰ | D¹¹ | D¹² | D¹³ | D¹⁴ | D¹⁵ | D¹⁶ |
|---|---|---|---|---|---|---|---|---|---|---|---|---|---|---|---|---|
| m | 4 | 5 | 4 | 4 | 4 | 5 | 5 | 5 | 4 | 4 | 4 | 5 | 5 | 5 | 4 | 5 |
| n | 4 | 4 | 5 | 4 | 4 | 5 | 4 | 4 | 5 | 5 | 4 | 5 | 5 | 4 | 5 | 5 |
| p | 4 | 4 | 4 | 5 | 4 | 4 | 5 | 4 | 5 | 4 | 5 | 5 | 4 | 5 | 5 | 5 |
| q | 4 | 4 | 4 | 4 | 5 | 4 | 4 | 5 | 4 | 5 | 5 | 4 | 5 | 5 | 5 | 5 |

In a preferred embodiment, the process according to the invention comprises the additional step of
(v) simulating movement of the one or more serial adsorbent beds C-Iₘ, the one or more serial adsorbent beds C-IIₙ, the one or more serial adsorbent beds C-IIIₚ, and the one or more serial adsorbent beds C-IV_{q} in opposite direction to flow direction of liquid such that
   at least one adsorbent bed, which was previously operated in zone I (C-Iₘ), is then operated in zone IV (C-IV_{q});
   at least one adsorbent bed, which was previously operated in zone II (C-IIₙ), is then operated in zone I (C-Iₘ);
   at least one adsorbent bed, which was previously operated in zone III (C-IIIₚ), is then operated in zone II (C-IIₙ); and
   at least one adsorbent bed, which was previously operated in zone IV (C-IV_{q}), is then operated in zone III (C-IIIₚ).

Preferably, the SMB comprises in a downstream arrangement with respect to a flow direction of liquid an inlet for a desorbent, previous to the zone I, an outlet for product saccharide, preferably allulose (extract), previous to the zone II, an inlet for educt saccharide, preferably fructose (feed), previous to the zone III, an outlet for residual educt saccharide, preferably fructose (raffinate), previous to the zone IV.

The operation of SMB and suitable designs of zones I, II, III and IV and of their connection with one another are known to the skilled person. In this regard, it may be referred to e.g. A. Rodrigues, Simulated Moving Bed Technology: Principles, Design and Process Applications, 1st ed., Butterworth-Heinemann, 2015; S. Ramaswamy, Separation and Purification Technologies in Biorefineries, 1st ed., Wiley 2013; T. Borren, Verfahrenstechnik 876, 2007, Untersuchungen zu chromatographischen Reaktoren mit verteilten Funktionalitäiten; and H. Schmidt-Traub, Preparative Chromatography, 2013, Viley-VCH.

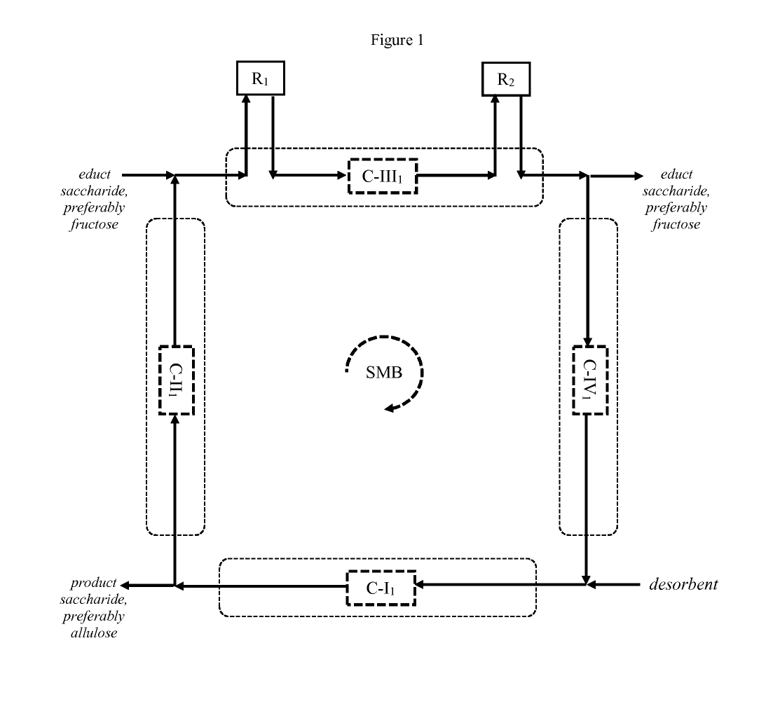

FIG. 1 schematically illustrates a preferred embodiment of the process according to the invention. Fructose is supplied in liquid form to a SMB comprising four zones (dotted rectangles) that are arranged in circular flow direction. (Fresh) educt saccharide, preferably fructose is supplied to zone III where it enters reactor R₁ in order to convert educt saccharide, preferably fructose to product saccharide, preferably allulose. The reaction product exits reactor R₂ and is supplied to a chromatography unit comprising adsorbent bed C-III₃ where product saccharide, preferably allulose and educt saccharide, preferably fructose are separated to a certain degree, typically not baseline separated. The first chromatographic fraction exiting the chromatography unit contains residual educt saccharide, preferably fructose and enters R₂ in order to convert educt saccharide, preferably fructose to product saccharide, preferably allulose. Due to the different retention times, the second chromatographic fraction containing product saccharide, preferably allulose has not yet exited the chromatography unit such that the reaction equilibrium in reactor R₂ is not influenced by the product saccharide, preferably allulose contained in the second chromatographic fraction, which subsequently enters and passes through reactor R₂. When the second chromatographic fraction enters reactor R₂, the first chromatographic fraction has preferably already exited reactor R₂. Residual educt saccharide, preferably fructose may be discharged and the residual liquid is supplied to zone IV, subsequently to zone I, followed by zone II, before it is returned to the inlet of (fresh) educt saccharide, preferably fructose. In a countercurrent manner, from time to time and in accordance with the desired switching time of the simulated movement of the bed, the adsorbent beds are simulated to move in a direction opposite to the flow direction of liquid, thereby allowing to discharge product saccharide, preferably allulose between zone I and zone II by means of a desorbent. Zones II and III essentially serve the purpose of separating residual educt saccharide, preferably fructose and product saccharide, preferably allulose from one another, whereas zones I and IV essentially serve the purpose of generating the adsorbent beds used in zones II and III prior to the next simulated move of the adsorbent beds.

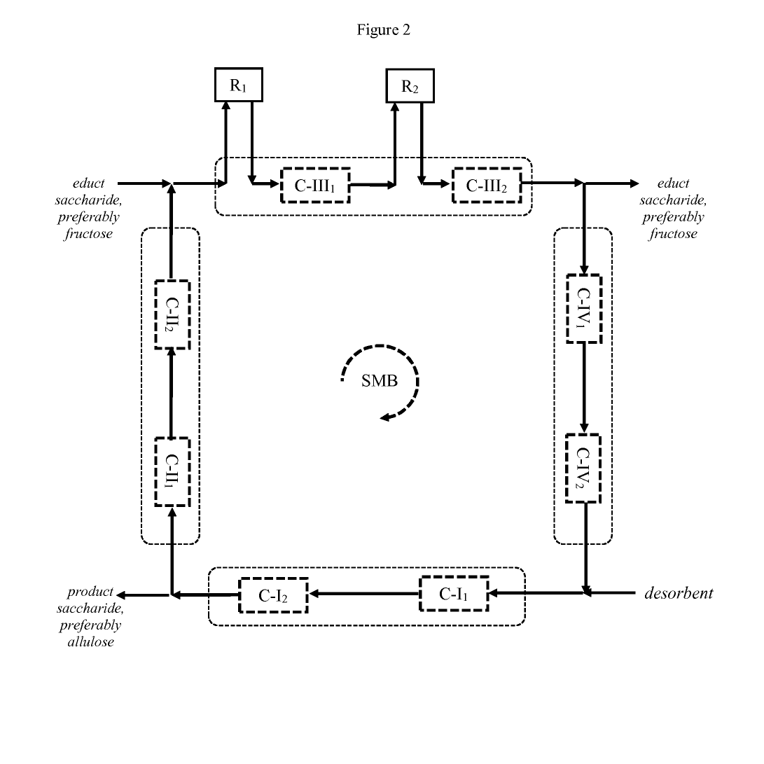

FIG. 2 schematically illustrates another preferred embodiment of the process according to the invention, wherein every zone comprises two adsorbent beds which may be contained in the same or in separate chromatography units.

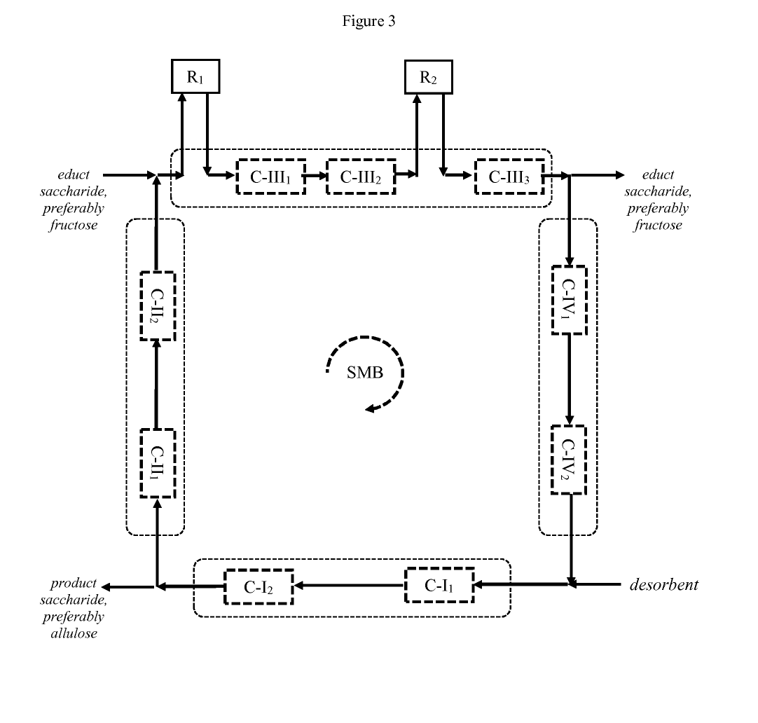

FIG. 3 schematically illustrates another preferred embodiment of the process according to the invention, wherein two adsorbent beds are arranged after reactor R₁ and before reactor R₂ in order to enhance chromatographic separation efficiency. Also in this embodiment, said two adsorbent beds may be contained in the same or in separate chromatography units, e.g. housings. FIG. 3 also illustrates an embodiment where the number of adsorbent beds differs in the various zones. While zone III comprises three adsorbent beds C-III₁, C-III₂ and C-III₃, zones I, II and IV each only comprise two adsorbent beds.

Preferably, the process according to the invention comprises the additional step of
(vi) recirculating residual educt saccharide, preferably fructose from the outlet for residual educt saccharide, preferably fructose to the inlet for educt saccharide, preferably fructose.

Preferably, the process according to the invention comprises the additional step of
(vii) filtering the liquid by means of a filter.

Preferably, the filter is operated in zone I, zone II, zone III and/or zone IV, as defined above.

Preferably, the process according to the invention comprises the additional step of
(viii) decoloring the liquid by means of a decolorizer.

Preferably, the decolorizer is operated in zone I, zone II, zone III and/or zone IV, as defined above.

Preferably, the process according to the invention comprises the additional step of
(ix) regulating the pH of the liquid by means of a pH regulator.

Preferably, the pH regulator is operated in zone I, zone II, zone III and/or zone IV, as defined above.

Preferably, the process according to the invention comprises the additional step of
(x) concentrating the liquid by means of a concentrator.

Preferably, the concentrator is operated in zone I, zone II, zone III and/or zone IV, as defined above.

Preferably, the process according to the invention comprises the additional step of
(xi) desalting the liquid by means of a desalter.

Preferably, the desalter is operated in zone I, zone II, zone III and/or zone IV, as defined above.

A second aspect of the invention in accordance with the Hashimoto process relates to apparatus for performing the process according to any of the preceding claims, comprising the following components in liquid flow communication
(I) a reactor R₁ which comprises an enzyme capable of converting educt saccharide, preferably fructose to product saccharide, preferably allulose;
(II) a first chromatography unit after reactor R₁ for separating product saccharide, preferably allulose from educt saccharide, preferably fructose; and
(III) a reactor R₂ after the first chromatography unit, wherein the reactor R₂ also comprises an enzyme capable of converting educt saccharide, preferably fructose to product saccharide, preferably allulose.

Preferably, the apparatus according to the invention additionally comprises in liquid flow communication (IV) a second chromatography unit after reactor R₂ for separating product saccharide, preferably allulose from educt saccharide, preferably fructose.

Preferably, the first chromatography unit and/or the second chromatography unit comprises an adsorbent bed comprising a calcium based resin.

Preferably, the reactor R₁ and/or the reactor R₂ is a chromatographic reactor or an immobilized column reactor.

Preferably, the first chromatography unit and the optionally present second chromatography unit are integrated in a simulated moving bed (SMB) separation system.

Preferably, the apparatus according to the invention additionally comprises in liquid flow communication
means for recirculating educt saccharide, preferably fructose to the reactor R₁; and/or
a filter; and/or
a decolorizer; and/or
a pH regulator; and/or
a concentrator; and/or
a desalter.

Another aspect of the invention in accordance with the Hashimoto process relates to of the apparatus according to the invention as described above for performing the process according to the invention as described above.

In another preferred embodiment, the educt saccharide, preferably fructose is converted to product saccharide, preferably allulose in a membrane reactor, wherein the enzyme is retained in the reactor by means of the membrane which, however, is permeable for the synthesized product saccharide, preferably allulose and for the non-converted educt saccharide, preferably fructose (residual starting material).

According to this embodiment, the membrane reactor may be coupled with an ultrafiltration device in which subsequent pre-purification step (d) may be performed.

Preferably, the membrane of the reactor has a cut-off of not more than 30 kDa, preferably not more than 25 kDa, more preferably not more than 20 kDa, most preferably not more than 15 kDa, and in particular not more than 10 kDa.

In still another preferred embodiment, the educt saccharide, preferably fructose is converted to product saccharide, preferably allulose under catalysis of immobilized enzymes or immobilized microorganisms.

In optional step (d) of the process according to the invention, the crude product composition provided in step (c) is pre-purified thereby providing a pre-purified product composition. Preferably, the pre-purified product composition is an aqueous liquid.

Preferably, step (d) involves substep (d₁), namely decoloring, preferably by means of active charcoal or decoloring resins that are specifically designed for that purpose and commercially available (e.g. Treverlite®, Chemra). The temperature for decoloring is preferably within the range of from 30° C. to 70° C.

Preferably, in addition to substep (d₁) or alternatively, step (d) involves substep (d₂), namely desalting, preferably by means of ion exchange resins. Preferably, substep (d₂) involves sequential desalting by means of differently charged ion exchange resins, e.g. commencing with cations exchangers, followed by anions exchangers, followed by mixed bed exchangers.

Alternatively or additionally, desalting may be achieved by reverse osmosis, electrodialysis, dialysis or chromatography.

Preferably, in addition to substeps (d₁) and/or (d₂) or alternatively, step (d) involves substep (d₃), namely filtration, preferably nanofiltration or ultrafiltration, thereby separating solids from the crude product composition. Ultrafiltration is preferred, especially when preceding enzymatic conversion step (c) is performed in a membrane reactor to which the ultrafiltration device may be coupled.

In optional step (e) of the process according to the invention, the crude product composition provided in step (c) or the pre-purified product composition provided in step (d) is concentrated thereby providing a concentrated product composition. Preferably, the concentrated product composition is an aqueous liquid.

Preferably, the concentration has the effect that the dry matter content relative to the total weight of the composition is relatively increased by at least 1 g dry matter per 100 g of the composition (concentrated product composition vs. crude product composition).

Concentration of the crude product composition provided in step (c) or of the pre-purified product composition provided in step (d) may be achieved by means of an evaporator, preferably at a temperature below 60° C. Suitable evaporators include but are not limited to rotation evaporators, plate evaporators, rising film plate evaporators (or vertical long tube evaporators), falling film evaporators, Robert evaporators and circulation evaporators, wherein in either case single step or multiple step evaporations are possible.

Preferably, evaporation is performed at elevated temperatures. Preferably, the temperature of the heating medium, e.g. steam, is within the range of from 100° C. to 150° C., more preferably 110° C. to 140° C., most preferably 120° C. to 130° C., whereas the product temperature is preferably within the range of from 30° C. to 59° C. It has been surprisingly found that at product temperatures of 60° C. and above, the product undesirably becomes colored, likely due to caramelization reactions. Preferably, evaporation is performed at reduced pressure, preferably at a vacuum within the range of from 1 mbar to 300 mbar.

Alternatively, concentration may be achieved by nanofiltration, preferably at a pressure within the range of from 20 bar to 60 bar, or by reverse osmosis, preferably at a pressure within the range of from 20 bar to 100 bar.

In either case, the crude product composition provided in step (c) or the pre-purified product composition provided in step (d) is preferably concentrated such that the final concentration of the dry matter including the product saccharide, preferably allulose in the thus provided concentrated product composition is suitable for subsequent processing, preferably in process step (f). Preferably, the concentration of dry matter, i.e. including product saccharide, preferably allulose and all other dissolved constituents but no water, in the thus provided concentrated product composition is within the range of from 40 to 80 wt.-%, based on the total weight of the concentrated product composition. In preferred embodiments, said concentration is within the range of 50±10 wt.-%, or 55±10 wt.-%, or 60±10 wt.-%.

In optional step (f) of the process according to the invention, the concentrated product composition provided in step (e) is purified by chromatography thereby providing a purified product saccharide composition. Preferably, the purified product saccharide composition is an aqueous liquid.

Chromatography may be performed continuously or batch-wise. When step (c) of the process according to the invention involves chromatographic reactors, preferably immobilized column reactors, combining biochemical conversion with chromatographic separation (Hashimoto process), the subsequent purifying by chromatography in step (f) is integrated in the (c).

Chromatography in step (f) essentially serves the purpose of separating product saccharide, preferably allulose and non-converted educt saccharide, preferably fructose (starting material) from one another. Thus, the purified product saccharide composition provided in step (f) has a substantially lower content of non-converted educt saccharide, preferably fructose than the product composition provided in step (c), the pre-purified product composition provided in step (d), and the concentrated product composition provided in step (e), respectively.

The thus separated non-converted educt saccharide, preferably fructose (starting material) may be recirculated to step (a) or to step (b) of the process according to the invention.

Chromatography is preferably performed as column chromatography at elevated pressure (MPLC or HPLC). Preferred methods of chromatography include but are not limited to batch chromatography, continuous chromatography, simulated moving bed (SMB) chromatography and sequential simulated moving bed chromatography (SSMB).

Suitable stationary phases for chromatographically separating product saccharide, preferably allulose and educt saccharide, preferably fructose from one another are known to the skilled person and commercially available. Preferred stationary phases are calcium based resins like DOWEX® MONOSPHERE 99 Ca, Lewatit® MDS 1368 Ca/320, Purolite® PCR642Ca.

Preferably, chromatography is performed at elevated temperature, preferably within the range of from 40° C. to 90° C., more preferably from 50° C. to 80° C., and most preferably from 60° C. to 75° C.

Preferably, the purity of product saccharide, preferably allulose in the thus provided purified product saccharide composition is within the range of from 65 wt.-% to 99 wt.-%, relative to the total content of dry matter, i.e. including product saccharide, preferably allulose and all other dissolved constituents but no water, that is contained in the purified product saccharide composition. In preferred embodiments, said purity is within the range of 75±10 wt.-%, or 80±10 wt.-%, or 85±10 wt.-%, or 90±10 wt.-%.

In optional step (g) of the process according to the invention, the purified product saccharide composition provided in step (f) is concentrated thereby providing a concentrated product saccharide composition. Preferably, the concentrated product saccharide composition is an aqueous liquid.

Preferably, the concentration has the effect that the dry matter content relative to the total weight of the composition is relatively increased by at least 1 g dry matter per 100 g of the composition (concentrated product saccharide composition vs. purified product saccharide composition).

Concentration of the purified product saccharide composition provided in step (f) may be achieved by means of an evaporator, preferably at a temperature below 60° C. Suitable evaporators include but are not limited to rotation evaporators, plate evaporators, rising film plate evaporators (or vertical long tube evaporators), falling film evaporators, Robert evaporators and circulation evaporators, wherein in either case single step or multiple step evaporations are possible.

Preferably, evaporation is performed at elevated temperatures. Preferably, the temperature of the heating medium, e.g. e.g. steam, is within the range of from 100° C. to 150° C., more preferably 110° C. to 140° C., most preferably 120° C. to 130° C., whereas the product temperature is preferably within the range of from 30° C. to 59° C. Preferably, evaporation is performed at reduced pressure, preferably at a vacuum within the range of from 1 mbar to 300 mbar.

Preferably, the concentration of dry matter, i.e. including product saccharide, preferably allulose and all other dissolved constituents but no water, in the thus provided concentrated product saccharide composition is within the range of from 40 wt.-% to 95 wt.-%, preferably 40 wt.-% to 70 wt.-%, or 70 wt.-% to 95 wt.-%, based on the total weight of the concentrated product saccharide composition. In preferred embodiments, said concentration is within the range of 50±10 wt.-%, or 55±10 wt.-%, or 60±10 wt.-%, or 65±10 wt.-%, or 70±10 wt.-%, or 75±10 wt.-%, or 80±10 wt.-%, or 85±10 wt.-%, or 90±10 wt.-%.

In mandatory step (h) of the process according to the invention, a liquid product saccharide product or a solid product saccharide product is provided.

Preferably, the purity of product saccharide, preferably allulose in the liquid or solid product saccharide product is within the range of from 65 wt.-% to 100 wt.-%, relative to the total content of dry matter, i.e. including product saccharide, preferably allulose and all other constituents but no water, that is contained in the liquid or solid product saccharide product. In preferred embodiments, said purity is within the range of 75±10 wt.-%, or 80±10 wt.-%, or 85±10 wt.-%, or 90±10 wt.-%.

When a liquid product saccharide product is provided, preferably an aqueous product saccharide, preferably allulose syrup, the liquid product saccharide product may essentially correspond to the purified product saccharide composition provided in step (f) or to the concentrated product saccharide composition provided in step (g).

Preferably, the concentration of product saccharide, preferably allulose in the liquid product saccharide product is at least 40 wt.-%, more preferably at least 45 wt.-%, still more preferably at least 50 wt.-%, yet more preferably at least 55 wt.-%, even more preferably at least 60 wt.-%, most preferably at least 65 wt.-% and in particular at least 70 wt.-%, relative to the total weight of the liquid product saccharide product. In a preferred embodiment, the concentration of dry matter in the liquid product saccharide product (syrup) is at least 60 wt.-%, more preferably at least 65 wt.-% and in particular at least 70 wt.-%, relative to the total weight of the liquid product saccharide product, and the content of product saccharide, preferably allulose is within the range of from 90 to 100 wt.-%, relative to the total content of dry matter.

The liquid product saccharide product is preferably filtered before packaging.

When a solid product saccharide product is provided, the solid product saccharide, preferably allulose is preferably isolated from solution, i.e. from the purified product saccharide composition provided in step (f) or the concentrated product saccharide composition provided in step (g), by precipitation, preferably by crystallization.

Preferably, the purity of product saccharide, preferably allulose, in the concentrated product saccharide composition from which the solid product saccharide product is provided by precipitation, is within the range of from 80 wt.-% to 100 wt.-%, relative to the total content of dry matter, i.e. including product saccharide, preferably allulose and all other constituents but no water, that is contained in the concentrated product saccharide composition from which the solid product saccharide product is provided by precipitation. In preferred embodiments, said purity is within the range of 75±10 wt.-%, or 80±10 wt.-%, or 85±10 wt.-%, or 90±10 wt.-%.

Preferably, the concentration of dry matter, i.e. including product saccharide, preferably allulose and all other dissolved constituents but no water, in the concentrated product saccharide composition from which the solid product saccharide product is provided by precipitation, is within the range of from 30 wt.-% to 99.9 wt.-%, based on the total weight of the concentrated product saccharide composition. In preferred embodiments, said concentration is within the range of 50±10 wt.-%, or 55±10 wt.-%, or 60±10 wt.-%, or 65±10 wt.-%, or 70±10 wt.-%, or 75±10 wt.-%, or 80±10 wt.-%, or 85±10 wt.-%, or 90±10 wt.-%.

Suitable devices for precipitation by crystallization are known to the skilled person and include but are not limited to cooling crystallizers, vacuum evaporation crystallizers, forced-circulation (FC), stirring containers, and internal guide sleeve crystallizers.

Suitable devices for grinding are known to the skilled person and include but are not limited to rotor mills, cutting mills, knife mills, mortar mills, disc mills, ball mills and jaw crushers.

Precipitation, preferably crystallization, may be performed e.g. as cooling crystallization or as vacuum evaporation crystallization with subsequent centrifugation, i.e. as cooling crystallization and subsequent centrifugation, or as evaporation crystallization and subsequent centrifugation.

Preferably, crystallization is performed as flash crystallization. Preferably, the vacuum in the flash crystallizator is within the range of from 1 mbar to 300 mbar.

Crystallization is preferably performed as suspension crystallization or as spontaneous crystallization or as flash crystallization.

Suspension crystallization according to the invention refers to crystallization due to controlled or uncontrolled oversaturation of a solution which contains the desired product (i.e. product saccharide, preferably allulose), solvent (e.g. water, ethanol, and the like) and may contain further constituents (carbohydrates, salts, and the like). The required oversaturation may be achieved by cooling and/or evaporation, optionally under vacuum.

Spontaneous crystallization according to the invention refers to crystallization, wherein a solution having a high concentration of the desired product (e.g. 95 wt.-% ds product saccharide, preferably allulose) is provided at a high temperature (e.g. 100 to 150° C.). Seed material of the desired product (product saccharide, preferably allulose) is added in solid form (crystalline, amorphous), while the solution is subjected to high shearing. In certain instances the addition of seed material may be omitted and crystallization is achieved by shearing only. Due to the high content of dry matter and the shearing the phase spontaneously changes from liquid to solid thereby releasing heat evaporating the water.

Flash crystallization according to the invention is achieved by spraying a heated undersaturated solution of the desired product (product saccharide, preferably allulose) in vacuum thereby providing a fine crystalline material. After a liquid/solid separation, the fine crystals may be agglomerated to one another.

When crystallization is performed as suspension crystallization, the purity of product saccharide, preferably allulose in the composition that is subjected to crystallization is preferably within the range of from 80 wt.-% to 100 wt.-%, relative to the total content of dry matter contained in said composition. Preferably, the content of dry matter is at least 60 wt.-%, relative to the total weight of said composition. Preferably, the composition is stirred at a revolution within the rage of from 1 rpm to 250 rpm. Preferably, the amount of seed crystals is within the range of from 0.001 wt.-% to 10 wt.-%, relative to the weight of the dry matter contained in said composition. Preferably, the seed crystals have an average particle size within the range of from 0.1 m to 200 am. Preferably, crystallization commences at a start temperature within the range of from 20° C. to 120° C., more preferably 30° C. to 65° C., and/or is terminated at an end temperature within the range of from 0° C. to 80° C., more preferably 25° C. to 40° C. Preferably, the cooling rate is within the range of from 5° C./h to 0.005° C./h, more preferably 1° C./h to 0.05° C./h. Preferably, crystallization is performed under vacuum, more preferably within the range of from 1 mbar to 200 mbar. Preferably, the average particle size of the crystalline product saccharide, preferably allulose product with within the range of from 10 μm to 20,000 μm, more preferably 10 μm to 1000 μm. Preferably, the precipitate is subjected to centrifugation and the amount of cover water that is added per volume of suspension is within the range of from 0 vol.-% to 70 vol.-%, relative to the volume of the solution after centrifugation.

When crystallization is performed as spontaneous crystallization, the purity of product saccharide, preferably allulose in the composition that is subjected to crystallization is preferably within the range of from 80 wt.-% to 100 wt.-%, relative to the total content of dry matter contained in said composition. Preferably, after evaporation, the content of dry matter is within the range of from 90 wt.-% to 99.9 wt.-%, relative to the total weight of said composition. Preferably, the product temperature during blending with the seed crystals is within the range of from 0° C. to 80° C. Preferably, blending is performed at a torque within the range of from 1 Nm to 5000 Nm. Preferably, the amount of seed crystals is within the range of from 1 wt.-% to 50 wt.-%, relative to the weight of the dry matter contained in said composition. Preferably, the average particle size of the crystalline product saccharide, preferably allulose product with within the range of from 10 μm to 2000 μm.

Alternatively, precipitation, preferably crystallization, may be achieved by means of a high shear blender, followed by subsequent classification, grinding and sieving.

Suitable devices for mixing and blending are known to the skilled person and include but are not limited to plow mixers, planetary mixers, and turbulizers.

Alternatively, precipitation, preferably crystallization, may be performed as spray drying, spray congealing, spray granulation or spray crystallization, or by means of a belt dryer or an infrared dryer.

Preferably, the temperature of the concentrated product saccharide composition (spray solution) from which the solid product saccharide product is provided by spray techniques such as spray drying or spray granulation is within the range of from 15° C. to 80° C.

Spray techniques such as spray drying or spray granulation are typically achieved by means of a spray tower. Preferably, the inlet temperature at the spray tower is within the range of from 40° C. to 200° C. Preferably, the mean drying residence time (volume per volume stream) is within the range of from 1 second to 3600 seconds. Preferably, the product temperature at the outlet of the spray tower is within the range of from 20° C. to 105° C. Preferably, the spray pressure is within the range of from 1 bar to 200 bar.

Suitable nozzels (jets) for spray techniques are known to the skilled person and include but are not limited to two component jets, hollow cone jets, multiple component jets, full cone jets, and flat stream jets.

When the solid product saccharide product is provided by spray granulation, the average particle size of the crystalline product saccharide, preferably allulose that is employed as seed material is preferably within the range of from 50 m to 500 am dₘᵢₙ-dₘₐₓ. Preferably, the ratio of the spray solution to the fluidized seed material is within the range of from 1% to 80%. For example, when the above ratio is 25% and 5 kg of seed material are fluidized, the spray solution amounts to 20 kg.

Suitable devices for granulation are known to the skilled person and include but are not limited to granulating plates, granulating drums, pressure agglomerizers, blending granulators, and melt granulators.

Product saccharide, preferably allulose from the production process having a particle size within the range of from 0.01 μm to 20,000 μm, preferably 0.05 μm to 2000 μm, is preferably supplied to centrifugation.

Suitable centrifuges that are capable of separating solids from liquids are known to the skilled person and include but are not limited to basket centrifuges. The centrifuges may be operated continuously or discontinuously. The rotational speed depends upon the fineness of the starting material. For purification of crystals, cover water may be used for rinsing. Other suitable rinsing liquids include but are not limited to methanol, ethanol, isopropanol, and the like.

When precipitation of the solid product saccharide product is achieved by spray drying, the average particle size of the thus provided product saccharide, preferably allulose particles is preferably within the range of from 50 μm to 500 μm dₘᵢₙ-dₘₐₓ.

When precipitation of the solid product saccharide product is achieved by spray granulation or flash-crystallization, the average particle size of the thus provided product saccharide, preferably allulose particles is preferably within the range of from 10 m to 20,000 μm dₘᵢₙ-dₘₐₓ.

In optional step (i′) of the process according to the invention, the solid product saccharide product provided in step (h) is (further) dried thereby providing a dried product saccharide product.

Suitable dryers include but are not limited to drum dryers, drying cabinets, vacuum dryers, spray dryers, infrared dryers, falling film dryers, fluidized bed dryers, vibration fluidized bed dryers, and revolver dryers.

Preferably, drying is performed at a temperature within the range of from 20° C. to 150° C. In preferred embodiments, drying is performed at a temperature within the range of 40±20° C., or 50±20° C., or 60±20° C., or 70±20° C., or 80±20° C., or 90±20° C., or 100±20° C., or 110±20° C., or 120±20° C., or 130±20° C.

The gas that is utilized in the drying process may be e.g. air, nitrogen or carbon dioxide which may optionally be pre-dried to a relative humidity within the range of from 0% to 20%.

The final moisture content of the dried product saccharide product is preferably within the range of from 0 wt.-% to 2 wt.-%, more preferably 0.001 wt.-% to 0.2 wt.-%.

After drying, the product saccharide, preferably allulose may be divided into fractions of different grain size. Suitable devices for screening (classifying) are known to the skilled person and include but are not limited to tumbling sieves, vibrational sieves, ultrasound sieves, rotational sieves, and the like. Screen cloth may be made from plastics or metal, may be woven, slotted, perforated or pierced.

Suitable mesh sizes include but are not limited to:

mesh size [μm]

100
200
250
315
400
500
610
730
800
910
1000
1150
1200
1400
1600
1800
2000
2200
2400
2800
3150
4000
5000
6100
7300
8000
10000
12500
15000
20000

Any intermediate mesh sizes are also possible.

In optional step (j) of the process according to the invention, the liquid product saccharide product provided in step (h) or the dried product saccharide product provided in step (i′) is packaged thereby providing a packaged product saccharide product.

Small packaging have preferred sizes within the range of from 50 g to 5000 g.

Suitable packaging machines are known to the skilled person and include but are not limited to machines based on volumetric dosing or gravimetric dosing by weighing mass differences. Dosing may be achieved e.g. by means of screws, vibrating chutes or conveyor belts.

Suitable packaging materials include but are not limited to paper, plastics and composite materials.

Suitable packaging include film tubing bags, composite tubing bags with weld seam, paper tubing bags with adhesive seam, and resealable bags. The bags may be designed as stand-up pouch, stand up cardboard box or chunk bottom bag. The foregoing may be equipped with an inner bag made from paper of plastic film.

Large packaging above 5 kg may also be made from paper, plastics or composites. Plastic films are preferably airtight, needled or pricked.

In optional step (k) of the process according to the invention, the packaged product saccharide product provided in step (j) is palletized thereby providing a palletized product saccharide product.

In optional step (l) of the process according to the invention, the packaged product saccharide product provided in step (j) or the palletized product saccharide product provided in step (k) is stored.

The packaged product saccharide product may be stored in bags, in big bags or as lose material in containers (silos). The storage temperature is preferably within the range of from 0° C. to 35° C., preferably about 20° C. The relative humidity at the storage is preferably within the range of from 0% to 80%, more preferably 30% to 50%.

The following examples further illustrate the invention but are not to be construed as limiting its scope.

### EXAMPLE 1—FULL PROCESS, MEMBRANE REACTOR

Crystalline fructose is employed as starting material for allulose production. The fructose is dissolved in water and the concentration is adjusted to 40 wt.-%, dry matter, relative to the total weight of the composition. The added water may be tap water, demineralized water, condensed water as provided in a subsequent step of the process, or a mixture of any of the foregoing. The pH value and electrolyte content is adjusted by adding appropriate buffers and salts.

The enzymatic conversion is performed in a membrane reactor (cut off 10 kDa) that is coupled to an ultrafiltration device. The enzymes in the reactor are freely dissolved, i.e. neither immobilized nor contained in microorganisms.

Purified lyophilized enzyme (D-tagatose 3-epimerase from Pseudomonas cichorii, expressed with E. coli JM109) or crude extract (cell free fermentation broth) is added to an aqueous solution of fructose at a concentration within the range of from 50 g/L to 500 g/L in 50 mM TRIS/HCl buffer and 1 mM MnCl₂. The pH value is adjusted to pH 7.5 or pH 9 by means of the required amount of HCl aq. and the stirred solution is incubated at 55° C. or 60° C. Depending upon the concentration of the fructose, after 1 hour a yield of 30% allulose relative to the employed fructose may be achieved:

| g/L | reaction time | | | | | |
|---|---|---|---|---|---|---|
| initial | 1 h | | | 24 h | | |
| fructose | fructose | allulose | yield | fructose | allulose | yield |
| [g/L] | [g/L] | [g/L] | [%] | [g/L] | [g/L] | [%] |
| 51 | 35 | 16 | 30 | 37 | 16 | 31 |
| 101 | 70 | 30 | 30 | 70 | 30 | 30 |
| 229 | 165 | 65 | 28 | 168 | 72 | 31 |
| 420 | 326 | 95 | 23 | 291 | 124 | 30 |

The composition containing the fructose is filtered through a filter (0.2 micrometer) and supplied to the membrane reactor. Fructose is converted to allulose by enzymatic catalysis for 36 hours at 30° C. The product is removed from the reactor by ultrafiltration thereby separating the carbohydrates (essentially allulose and residual fructose) from the enzymes which in turn are recycled to the membrane reactor for reuse.

The composition is pre-purified. Decoloring is achieved by means of a decoloring column or by means of active charcoal, in either case at a temperature within the range of from 30° C. to 70° C. Desalting is achieved by means of ion exchange resins, commencing with cations exchangers, followed by anions exchangers, followed by mixed bed exchangers.

The thus provided composition is concentrated by means of an evaporator at a temperature of below 60° C. and the concentration of dry matter is adjusted to a concentration within the range of from 40 wt.-% to 70 wt.-%, relative to the total weight of the composition. The evaporator is selected from rising film plate evaporator (or vertical long tube evaporator), falling film evaporator, Robert evaporator and circulation evaporator, wherein in either case single step or multiple step evaporations are possible. Allulose and residual fructose are separated from one another by chromatography. The chromatography is selected from batch chromatography, continuous chromatography, simulated moving bed (SMB) chromatography and sequential simulated moving bed (SMB) chromatography (SSMB).

The thus provided composition is again concentrated by means of an evaporator at a temperature of below 60° C. and the concentration of dry matter is adjusted to a concentration within the range of from 70 wt.-% to 95 wt.-%, relative to the total weight of the composition. The evaporator is selected from rising film plate evaporator (or vertical long tube evaporator), falling film evaporator, Robert evaporator and circulation evaporator, wherein in either case single step or multiple step evaporations are possible.

From the thus provided composition allulose is provided as a solid material by cooling crystallization and subsequent centrifugation, or by evaporation crystallization and subsequent centrifugation, or by high shear blending and subsequent grinding and sieving, or by spray drying, or by spray granulation, or by spray crystallization, or by means or a belt dryer, or by means of an infrared dryer. The allulose is then (further) dried by means of a drum dryer, or by means of a fluidized bed dryer, or by means of a vibration fluidized bed dryer, or by means of a revolver dryer. The solid allulose is them packaged in bags and palletized.

### EXAMPLE 2—FULL PROCESS, MEMBRANE REACTOR

Fructose syrup is employed as starting material for allulose production. In accordance with Example 1,
the pH value and electrolyte content is adjusted by adding appropriate buffers and salts or titration during reaction;
the enzymatic conversion is performed in a membrane reactor;
decoloring is achieved by means of a decoloring column or by means of active charcoal;
allulose and residual fructose are separated from one another by chromatography; and
concentrating is achieved by means of an evaporator involving multiple step evaporations.

The liquid product saccharide product is then filtered and the allulose concentration is optionally adjusted by adding water. The liquid product saccharide product is packaged in bags and stored.

### EXAMPLE 3—FULL PROCESS, HASHIMOTO

In accordance with Example 2,
fructose syrup is employed as starting material for allulose production; and
the pH value is adjusted by adding appropriate salts.

The enzymatic conversion is performed according to a Hashimoto process, i.e. in a chromatographic reactor already providing a product saccharide composition from which the residual non-converted fructose has been separated by chromatography.

In accordance with Examples 1 and 2, concentrating is achieved by means of an evaporator.

Precipitation of allulose from the concentrated aqueous solution is achieved by cooling crystallization.

### EXAMPLE 4—FULL PROCESS, IMMOBILIZED ENZYME

Fructose that was provided as a co-product from another process is employed as starting material. Said another process is in accordance with WO 2016/038142. In accordance with Examples 1 and 2, the pH value and electrolyte content is adjusted by adding appropriate buffers and salts.

The enzymatic conversion is catalyzed by immobilized enzyme.

In accordance with Example 1,
decoloring is achieved by means of a decoloring column or by means of active charcoal;
desalting is achieved by means of ion exchange resins;
concentrating is achieved by means of an evaporator;
allulose and residual fructose are separated from one another by chromatography; and
concentrating is achieved by means of another evaporator.

Precipitation of allulose from the concentrated aqueous solution is achieved by spray drying.

### EXAMPLE 5—FULL PROCESS, MEMBRANE REACTOR

A glucose/fructose syrup is employed as starting material for allulose production.

In accordance with Example 1,
the concentration is adjusted;
the enzymatic conversion is performed in a membrane reactor;
decoloring is achieved by means of a decoloring column or by means of active charcoal;
desalting is achieved by means of ion exchange resins;
concentrating is achieved by means of an evaporator;
allulose and residual fructose are separated from one another by chromatography; and
concentrating is achieved by means of another evaporator.

Precipitation of allulose from the concentrated aqueous solution is achieved by means of a high shear blender.

### EXAMPLE 6—FULLY PROCESS, MEMBRANE REACTOR

In accordance with Example 5, a glucose/fructose syrup is employed as starting material.

In accordance with Example 1,
the pH value and electrolyte content is adjusted by adding appropriate buffers and salts;
the enzymatic conversion is performed in a membrane reactor;
decoloring is achieved by means of a decoloring column or by means of active charcoal;
allulose and residual fructose are separated from one another by chromatography; and
concentrating is achieved by means of an evaporator involving multiple step evaporations.

Precipitation of allulose from the concentrated aqueous solution is achieved by means of evaporation crystallization.

### EXAMPLE 7—SPRAY GRANULATION, BOTTOM SPRAY

A spray tower was used having a total height of 2 m, a maximal diameter of 0.75 m, a conically tapered product room (height 1 m), and a volume of about 0.6 m³. 5 kg of allulose having an average particle size of 100 m were employed as seed material. The volume flow was guided from the bottom to the top and adjusted to 300 m³/h resulting in an average minimal residence time of about 7 seconds. The inlet temperature of the air flow was adjusted to a temperature between 140° C. and 160° C. and the product temperature was at most 95° C.

An aqueous allulose solution having a content of dry matter of 65 wt.-% and a purity of 95 wt.-%, relative to the total content of dry matter, was sprayed at room temperature and at a pressure of 5 bar through a bottom spray nozzle (two component jet) in coflow with the supplied air. The spray solution to seed material ratio was 25%.

The product was continuously discharged by means of a zig-zac-separator at a counter pressure of 0.4 bar. The product had an average particle size within the range of from 120 m to 140 am and a moisture content of below 1 wt.-%. The product was free-flowing.

### EXAMPLE 8—SPRAY GRANULATION, BOTTOM SPRAY

In accordance with Example 7, a spray tower was used having a total height of 2 m, a maximal diameter of 0.75 m, a conically tapered product room (height 1 m), and a volume of about 0.6 m³. 5 kg of allulose having an average particle size of 200 μm were employed as seed material. The volume flow was guided from the bottom to the top and adjusted to 450 m³/h resulting in an average minimal residence time of about 5 seconds. The inlet temperature of the air flow was adjusted to a temperature between 140° C. and 160° C. and the product temperature was at most 95° C.

An aqueous allulose solution having a content of dry matter of 70 wt.-% and a purity of 99 wt.-%, relative to the total content of dry matter, was sprayed at room temperature and at a pressure of 5 bar through a bottom spray nozzle (two component jet) in coflow with the supplied air. The spray solution to seed material ratio was 20%.

The product was continuously discharged by means of a zig-zac-separator at a counter pressure of 0.6 bar. The product had an average particle size within the range of from 250 μm to 270 μm and a moisture content of below 1 wt.-%. The product was free-flowing.

### EXAMPLE 9—SPRAY GRANULATION, BOTTOM SPRAY

In accordance with Example 7 and 8, a spray tower was used having a total height of 2 m, a maximal diameter of 0.75 m, a conically tapered product room (height 1 m), and a volume of about 0.6 m³. 5 kg of allulose having an average particle size of 350 μm were employed as seed material. The volume flow was guided from the bottom to the top and adjusted to 600 m³/h resulting in an average minimal residence time of about 4 seconds. The inlet temperature of the air flow was adjusted to a temperature between 140° C. and 160° C. and the product temperature was at most 95° C.

An aqueous allulose solution having a content of dry matter of 70 wt.-% and a purity of 95 wt.-%, relative to the total content of dry matter, was sprayed at room temperature and at a pressure of 5 bar through a bottom spray nozzle (two component jet) in coflow with the supplied air. The spray solution to seed material ratio was 30%.

The product was continuously discharged by means of a zig-zac-separator at a counter pressure of 0.8 bar. The product had an average particle size within the range of from 350 m to 400 μm and a moisture content of below 3 wt.-% and exhibited adherences of syrup.

### EXAMPLE 10—SPRAY GRANULATION, TOP SPRAY

In accordance with Examples 7 to 9, a spray tower was used having a total height of 2 m, a maximal diameter of 0.75 m, a conically tapered product room (height 1 m), and a volume of about 0.6 m³. 5 kg of allulose having an average particle size of 100 μm were employed as seed material. The volume flow was guided from the bottom to the top and adjusted to 300 m³/h resulting in an average minimal residence time of about 7 seconds. The inlet temperature of the air flow was adjusted to a temperature between 140° C. and 160° C. and the product temperature was at most 95° C.

An aqueous allulose solution having a content of dry matter of 65 wt.-% and a purity of 95 wt.-%, relative to the total content of dry matter, was sprayed at room temperature and at a pressure of 5 bar through a top spray nozzle (two component jet) in coflow with the supplied air. The spray solution to seed material ratio was 25%.

The product had an average particle size within the range of from 100 μm to 120 μm and a moisture content of below 1 wt.-%. The product was free-flowing.

### EXAMPLE 11—SPRAY DRYING, TOP SPRAY

A spray tower was used having a total height of 2 m, a maximal diameter of 0.75 m, a conically tapered product room (height 1 m), and a volume of about 0.6 m³. The volume flow was guided from the top to the bottom and adjusted to 600 m³/h resulting in an average minimal residence time of about 4 seconds. The inlet temperature of the air flow was adjusted to a temperature between 180° C. and 220° C.

An aqueous allulose solution having a content of dry matter of 65 wt.-% and a purity of 99 wt.-%, relative to the total content of dry matter, was heated to 50° C. and sprayed at a pressure of 40 bar through a top spray nozzle (two component jet) in coflow with the supplied air.

The product had an average particle size within the range of from 80 μm to 120 μm.

### EXAMPLE 12—SPRAY DRYING, TOP SPRAY

In accordance with Example 11, a spray tower was used having a total height of 2 m, a maximal diameter of 0.75 m, a conically tapered product room (height 1 m), and a volume of about 0.6 m³. The volume flow was guided from the top to the bottom and adjusted to 300 m³/h resulting in an average minimal residence time of about 7 seconds. The inlet temperature of the air flow was adjusted to a temperature between 180° C. and 220° C.

An aqueous allulose solution having a content of dry matter of 65 wt.-% and a purity of 99 wt.-%, relative to the total content of dry matter, was heated to 50° C. and sprayed at a pressure of 5 bar through a top spray nozzle (two component jet) in coflow with the supplied air.

The product had an average particle size within the range of from 150 m to 200 μm.

### EXAMPLE 13—FLASH CRYSTALLIZATION

A crystallization container having a conically tapered bottom was used at a vacuum of 100 mbar. An aqueous allulose solution having a content of dry matter of 80 wt.-% and a purity of 99 wt.-%, relative to the total content of dry matter, was heated to 50° C. and sprayed at a pressure of 50 bar through a top spray nozzle (hollow cone jet). In the crystallizer an oversaturated solution was present having a content of dry matter of 85 wt.-% and a purity of 99 wt.-%, relative to the total content of dry matter, at a temperature of 50° C. By spraying the allulose solution a product with an average particle size within the range of from 30 μm to 90 μm was provided as a suspension at the bottom.

The suspension was centrifuged at 6000 rpm by adding desalted water for 10 minutes. The separated crystals were granulated in a granulating drum by spraying allulose solution (50° C.) having a content of dry matter of 70 wt.-% and a purity of 99 wt.-%, relative to the total content of dry matter. The provided product had an average particle size of 200 μm.

### EXAMPLE 14—CONCENTRATING BY MEANS OF A ROTARY EVAPORATOR

The flask of a rotary evaporator was filled with 5500 g of an allulose-solution (69 wt.-% dry matter; purity=95 wt.-%). The allulose solution was evaporated at a water bath temperature of 80° C. at 70 rpm under a vacuum of 18 mbar yielding a dry matter of 85 wt.-%.

### EXAMPLE 15—CONCENTRATING BY MEANS OF A ROTARY EVAPORATOR

The flask of a rotary evaporator was filled with 5500 g of an allulose-solution (45 wt.-% dry matter; purity=99 wt.-%). The allulose solution was evaporated at a water bath temperature of 80° C. at 70 rpm under a vacuum of 12 mbar yielding a dry matter of 86.5 wt.-%.

### EXAMPLE 16—EVAPORATION BY MEANS OF A RISING FILM PLATE EVAPORATOR

A rising film plate evaporator was continuously fed with an allulose solution (35 wt.-% dry matter; 95 wt.-% purity) and evaporated in two stages. The steam pressure of the first stage was 3 bara (134° C.) and the product space was operated at a vacuum of 30 mbar. Upon exiting the first stage, the product temperature was 56° C. and the dry matter content 75 wt.-%. Upon exiting the second stage, the product temperature was 59° C. and the dry matter content 85 wt.-%.

### EXAMPLE 17—PREPARATION OF SEED CRYSTALS

A purified allulose solution containing 3585 g allulose was concentrated to a dry matter content of 79.4 wt.-%. The solution was stirred at 40 rpm in a crystallizer at a temperature of 32° C. A seed crystal solution of allulose crystals (Sigma-Aldrich; purity≥95 wt.-%) in ethanol (Merck, p.a.) was added at a ratio of 0.7 wt.-% (g seed crystals/g allulose in solution). The temperature of the solution was decreased by 2° C. at a rate of 1° C./h. After 48 hours, seed crystals could be separated by centrifugation. The crystals were dried in a fluidized bed dryer at a product temperature of 55° C. and had an average particle size within the range of from 0.1 m to 200 m.

### EXAMPLE 18—PREPARATION OF SEED CRYSTALS

The crystals according to Example 17 were classified by sieve separation of suitable mesh size. The classified crystals were combined to a slurry in ethanol. The weight ratio of dry matter (allulose) and liquid phase (ethanol, p.a.; Merck) was 1:4.

### EXAMPLE 19—COOLING CRYSTALLIZATION

A purified allulose solution having a purity of 95 wt.-% and containing 3733 g allulose was evaporated to a dry matter content of 86 wt.-%. The solution was stirred at 40 rpm in a crystallizer at a temperature of 50° C. The tempered solution was seeded with the suspension of Example 18. The utilized crystal fraction was 50 μm to 120 μm. The seed crystals amounted to a content of 0.3 wt.-% (seed crystals/g allulose solution).

The revolution speed was transiently increased in order to distribute the seed crystals homogenously in the solution and then reset to 40 rpm. The crystallization was performed at a linear cooling gradient of 0.085° C./h and was terminated at 30° C. The suspension was separated for 20 minutes by means of a centrifuge at 8000 rpm. Cover water (desalted) was added at a ratio of 20 vol.-% (desalted water:volume of suspension). 1980 g crystalline allulose were provided corresponding to a yield of 53 wt.-%. The size fraction was 50 μm to 150 μm (d15 to d85).

### EXAMPLE 20—COOLING CRYSTALLIZATION

A purified allulose solution having a purity of 99 wt.-% and containing 4230 g allulose was evaporated to a dry matter content of 82.5 wt.-%. The solution was stirred at 10 rpm in a crystallizer at a temperature of 45° C. The tempered solution was seeded with the suspension of Example 18. The utilized crystal fraction was 40 μm to 100 μm. The seed crystals amounted to a content of 1 wt.-% (seed crystals/g allulose solution).

The revolution speed was transiently increased in order to distribute the seed crystals homogenously in the solution and then reset to 10 rpm. The crystallization was performed at a linear cooling gradient of 0.16° C./h and was terminated at 35° C. The suspension was separated for 10 minutes by means of a centrifuge at 8000 rpm. Cover water (desalted) was added at a ratio of 10 vol.-% (desalted water:volume of suspension). 2370 g crystalline allulose were provided corresponding to a yield of 56 wt.-%. The size fraction was 300 μm to 400 μm (d15 to d85).

### EXAMPLE 21—COOLING CRYSTALLIZATION

A purified allulose solution having a purity of 90 wt.-% and containing 3890 g allulose was evaporated to a dry matter content of 86.5 wt.-%. The solution was stirred at 20 rpm in a crystallizer at a temperature of 55° C. The solution was cooled to 52° C. at a cooling rate of 1° C./h. The tempered solution was seeded with the suspension of Example 18. The utilized crystal fraction was 40 μm to 100 μm. The seed crystals amounted to a content of 0.5 wt.-% (seed crystals/g allulose solution).

The crystallization was performed at a linear cooling gradient of 0.1° C./h and was terminated at 25° C. The suspension was separated for 10 minutes by means of a centrifuge at 8000 rpm. Cover water (desalted) was added at a ratio of 5 vol.-% (desalted water:volume of suspension). 1560 g crystalline allulose were provided corresponding to a yield of 40 wt.-%. The size fraction was 50 μm to 120 μm (d15 to d85).

### EXAMPLE 21—EVAPORATION CRYSTALLIZATION

A purified allulose solution having a purity of 95 wt.-% and containing 7040 g allulose was evaporated to a dry matter content of 86.5 wt.-%. The solution was stirred at 20 rpm in an evaporation crystallizer at a temperature of 55° C. After temperature was equilibrated, a vacuum was set to 60 mbar.

The solution was seeded with the suspension of Example 18. The utilized crystal fraction was 40 μm to 100 μm. The seed crystals amounted to a content of 0.5 wt.-% (seed crystals/g allulose solution). The solution was concentrated by evaporation. The decrease of saturation was controlled by refractometry. The decrease should not exceed 2-3% (by refractometry) and by continuous evaporation approximate the initial value. In case that the saturation was to fast, the vacuum was reduced in order to avoid fine particle formation. The suspension was separated for 15 minutes by means of a centrifuge at 8000 rpm. Cover water (desalted) was added at a ratio of 20 vol.-% (desalted water:volume of suspension).

### EXAMPLE 22—EVAPORATION CRYSTALLIZATION WITH AFTER TREATMENT

A purified allulose solution having a purity of 97 wt.-% and containing 5230 g allulose was evaporated to a dry matter content of 86 wt.-%. The solution was stirred in an evaporation crystallizer at a temperature of 50° C. After temperature was equilibrated, a vacuum was set to 70 mbar.

The solution was seeded with the suspension of Example 18. The utilized crystal fraction was 50 m to 120 μm. The seed crystals amounted to a content of 0.4 wt.-% (seed crystals/g allulose solution). The solution was concentrated by evaporation. The decrease of saturation was controlled by refractometry. The decrease should not exceed 2-3% (by refractometry) and by continuous evaporation approximate the initial value. In case that the saturation was to fast, the vacuum was reduced in order to avoid fine particle formation.

In order to operate the process continuously, after reduction by 100 g condensate the corresponding weight of allulose solution was added (purity 97%, dry matter content 85 wt.-%). The crystallization was terminated when no significant decrease of saturation could be observed by refractometry.

The suspension was separated for 15 minutes by means of a centrifuge at 8000 rpm. Cover water (desalted) was added at a ratio of 35 vol.-% (desalted water:volume of suspension).

### EXAMPLE 23—SPONTANEOUS CRYSTALLIZATION

A purified allulose solution having a purity of 99 wt.-% and containing 2140 g allulose was evaporated to a dry matter content of 99 wt.-%. The solution was stirred in an mixer at 4000 rpm and at 80° C. After temperature was equilibrated, a vacuum was set to 70 mbar. 212 g crystalline allulose were added to the solution (10%). The solution was stirred for 30 minutes under these conditions. In the course of the stirring operation, a significant turbidity could be observed. After termination of the mixing operation, the mixture was distributed on a drying tray as flat as possible and dried in a drying cabinet at 50° C. The dried mass having a residual moisture content of less than 1 wt.-% was ground by means of a knife mill. The particles size was 50 μm to 120 μm.

### EXAMPLE 24—HASHIMOTO PROCESS

In the following table, preferred conditions of the Hashimoto process according to the invention are compiled:

| description | unit | preferred numerical range |
|---|---|---|
| Simulated Moving Bed | | |
| number of adsorbent beds | [—] | 4-24 |
| number of adsorbent beds per zone | [—] | 1-20 |
| ratio diameter/length adsorbent bed | [—] | 0.01-10 |
| volume adsorbent bed | [m³] | 0.00001-10 |
| temperature adsorbent bed | [° C.] | 30-90 (more preferably 40-80) (still more preferably 60-80) |
| pressure adsorbent bed | [bar] | 0.5-10 (more preferably 2-8) (still more preferably 3-5) |
| switching time | [s] | 10-10¹⁰ |
| throughput adsorbent bed | [m³/h] | 0.0001-10 |
| position circulatory pump | moving along or stationary | |
| zone operation SMB | 3 or 4 zones | |
| distribution system | fractal distributer, frit, round blank with welded sieve | |
| Reactor Part | | |
| number of reactors | [—] | 1-16 |
| feed concentration | [g/L] | 0.001-1000 |
| ratio adsorbent bed/reactor | [—] | 2-24 (more preferably 4) |
| volume reactor | [m³] | 0.00001-10 |
| temperature reactor | [° C.] | 10-90 (more preferably 40-70) (still more preferably 50-60) |
| pressure reactor | [bar] | 0.5-4 (more preferably 1-3) (still more preferably 1.5-2) |
| residence time reactor | [h] | 0.05-24 |

| | |
|---|---|
| position reactor | raffinate (fructose)- or extract (allulose) side (zone) |
| Additional Procedures | |
| concentrator | rotation evaporator, plate evaporator, rising film plate evaporator, |
| | falling film evaporator, Robert evaporator, or circulation evaporator |
| decolorizer | active charcoal, decolorizer resins |
| desalting | reverse osmosis, electrodialysis, dialysis, chromatography |
| pH regulator | addition of salts |

### EXAMPLE 25—HASHIMOTO PROCESS

In the following table, preferred conditions of the Hashimoto process according to the invention are compiled:

| | | preferred |
|---|---|---|
| description | unit | numerical range |
| Simulated Moving Bed | | |
| number of adsorbent beds | [—] | 16 |
| number of adsorbent beds per zone | [—] | 8-4-4 |
| ratio diameter/length adsorbent bed | [—] | 0.135 |
| volume adsorbent bed | [m³] | 0.000015 |
| temperature adsorbent bed | [° C.] | 50 |
| pressure adsorbent bed | [bar] | 2 |
| switching time | [s] | 120 |
| throughput adsorbent bed | [m³/h] | 0.000144 |
| position circulatory pump | moving along | |
| zone operation SMB | 3 | |
| distribution system | frit | |
| Reactor Part | | |
| number of reactors | [—] | 7 |
| feed concentration | [mol/m³] | 100 |
| ratio adsorbent bed/reactor | [—] | 2.28 |
| volume reactor | [m³] | 0.000015 |
| temperature reactor | [° C.] | 50 |
| pressure reactor | [bar] | 2 |
| residence time reactor | [h] | 0.104 |
| position reactor | extract (allulose) side (zone) | |
| reactor type | immobilized enzyme reactor | |
| Additional Procedures | | |
| concentrator | | |
| decolorizer | | |
| desalting | | |
| pH regulator | | |
| immobilization | | |
| Results | | |
| purity extract (allulose) | [%] | 89.1 |
| purity raffinate (fructose) | [%] | |
| yield | [%] | 80.1 |
| productivity | [kg/m³h] | 8500 |

### EXAMPLE 26—HASHIMOTO PROCESS

In the following table, preferred conditions of the Hashimoto process according to the invention are compiled:

| | | preferred |
|---|---|---|
| description | unit | numerical range |
| Simulated Moving Bed | | |
| number of adsorbent beds | [—] | 24 |
| number of adsorbent beds per zone | [—] | 6-6-6-6 |
| ratio diameter/length adsorbent bed | [—] | 0.25 |
| volume adsorbent bed | [m³] | 0.3927 |
| temperature adsorbent bed | [° C.] | 70 |
| pressure adsorbent bed | [bar] | 3 |
| switching time | [s] | 1500 |
| throughput adsorbent bed | [m³/h] | 0.750 |
| position circulatory pump | moving along | |
| zone operation SMB | 4 | |
| distribution system | round blank with sieve | |
| Reactor Part | | |
| number of reactors | [—] | 4 |
| feed concentration | [kg/m³] | 300 |
| ratio adsorbent bed/reactor | [—] | 6 |
| volume reactor | [m³] | 0.4 |
| temperature reactor | [° C.] | 60 |
| pressure reactor | [bar] | 2 |
| residence time reactor | [h] | 0.533 |
| position reactor | raffinate (fructose) side (zone) | |
| reactor type | immobilized enzyme reactor | |
| Additional Procedures | | |
| concentrator | | |
| decolorizer | Treverlite ®. Chemra | |
| desalting | | |
| pH regulator | | |
| immobilization | | |
| Results | | |
| purity extract (allulose) | [%] | 92.5 |
| purity raffinate (fructose) | [%] | 90.2 |
| yield | [%] | 88.8 |
| productivity | [kg/m³h] | 9870 |

### EXAMPLE 27—HASHIMOTO PROCESS

In the following table, preferred conditions of the Hashimoto process according to the invention are compiled:

| | | preferred |
|---|---|---|
| description | unit | numerical range |
| Simulated Moving Bed | | |
| number of adsorbent beds | [—] | 24 |
| number of adsorbent beds per zone | [—] | 2-2-2 |
| ratio diameter/length adsorbent bed | [—] | 0.06 |
| volume adsorbent bed | [m³] | 0.0002 |
| temperature adsorbent bed | [° C.] | 50 |
| pressure adsorbent bed | [bar] | 3 |
| switching time | [s] | 860 |
| throughput adsorbent bed | [m³/h] | 0.0002 |
| position circulatory pump | moving along | |
| zone operation SMB | 3 | |
| distribution system | frit | |
| Reactor Part | | |
| number of reactors | [—] | 1 |
| feed concentration | [kg/m³] | 300 |
| ratio adsorbent bed/reactor | [—] | 6 |
| volume reactor | [m³] | 0.0002 |
| temperature reactor | [° C.] | 50 |
| pressure reactor | [bar] | 2 |
| residence time reactor | [h] | 1 |
| position reactor | extract (allulose) side (zone) | |
| reactor type | immobilized enzyme reactor | |
| Additional Procedures | | |
| concentrator | | |
| decolorizer | | |
| desalting | | |
| pH regulator | | |
| immobilization | | |
| Results | | |
| purity extract (allulose) | [%] | 74.0 |
| purity raffinate (fructose) | [%] | — |
| yield | [%] | 67.0 |
| productivity | [kg/m³h] | 4000 |

1: A process for the synthesis of product saccharide in at least two reactors R₁ and R₂, the method comprising the steps of
(i) supplying a liquid comprising educt saccharide to the reactor R₁ and converting a portion of the educt saccharide to product saccharide under enzymatic catalysis thereby providing a liquid comprising product saccharide and residual educt saccharide;
(ii) separating at least a portion of the product saccharide from the residual educt saccharide of step (i) by liquid chromatography thereby providing
a first chromatographic fraction comprising residual educt saccharide and optionally product saccharide; and
a second chromatographic fraction comprising product saccharide and optionally residual educt saccharide; and
(iii) supplying the first chromatographic fraction of step (ii) to the reactor R₂ and converting at least a portion of the residual educt saccharide to product saccharide under enzymatic catalysis.

2-4. (canceled)

5: The process according to claim 1, wherein
(i) the educt saccharide is glucose, the product saccharide is fructose, and the conversions according to step (i) and/or step (iii) are performed under enzymatic catalysis by glucose-fructose-epimerase; or
(ii) the educt saccharide is fructose, the product saccharide is tagatose, and the conversions according to step (i) and/or step (iii) are performed under enzymatic catalysis by tagatose-3-epimerase; or
(iii) the educt saccharide is a monosaccharide, preferably galactose, the product saccharide is a monosaccharide, preferably tagatose, and the conversions according to step (i) and/or step (iii) are performed under enzymatic catalysis by tagatose-3-epimerase; or
(iv) the educt saccharide is a mixture of glucose-1-phosphate and glucose, the product saccharide is cellobiose, and the conversions according to step (i) and/or step (iii) are performed under enzymatic catalysis by cellobiose phosphorylase; or
(v) the educt saccharide is a mixture of glucose-1-phosphate and fructose, the product saccharide is sucrose, and the conversions according to step (i) and/or step (iii) are performed under enzymatic catalysis by sucrose phosphorylase.

6-9. (canceled)

10: The process according to claim 1, wherein the second chromatographic fraction is supplied to the reactor R₂ after the first chromatographic fraction.

11-17. (canceled)

18: The process according to claim 1, wherein reactor R₁ and/or reactor R₂ is a membrane reactor or a chromatographic reactor, preferably an immobilized column reactor.

19: The process according to claim 1, which is performed continuously.

20: The process according to claim 1, wherein the liquid chromatography of step (ii) is integrated in a simulated moving bed (SMB).

21: The process according to claim 20, wherein liquid flows through the SMB in a flow direction and an adsorbent bed is simulated to move in opposite direction.

22: The process according to claim 20, wherein the SMB comprises four zones I to IV, wherein liquid is cycled through zones I to IV and wherein with respect to flow direction of liquid zone IV is downstream zone III, zone III is downstream zone II, zone II is downstream zone I, and zone I is downstream zone IV.

23. (canceled)

24: The process according to claim 20, wherein the SMB comprises
a zone I comprising one or more serial adsorbent beds C-Iₘ, wherein index m is an integer of at least 1;
a zone II comprising one or more serial adsorbent beds C-IIₙ, wherein index n is an integer of at least 1;
a zone III comprising the reactor R₁ for the conversion of step (i), the reactor R₂ for the conversion of step (iii), and one or more serial adsorbent beds C-IIIₚ, wherein index p is an integer of at least 1; wherein with respect to flow direction of liquid (eluent) at least one of said adsorbent beds C-IIIₚ is arranged downstream the reactor R₁ and upstream the reactor R₂; and
a zone IV comprising one or more serial adsorbent beds C-IV_{q}, wherein index p is an integer of at least 1.

25: The process according to claim 24, wherein indices m, n, p and q are independently of one another within the range of from 1 to 12.

26: The process according to claim 24, wherein at least one of indices m, n, p and q is greater than 1.

27: The process according to claim 24, wherein indices m, n, p and q are identical.

28: The process according to claim 24, wherein indices m, n, p and q are not identical.

29-31. (canceled)

32: The process according to claim 1, which comprises the additional step of filtering the liquid by means of a filter.

33. (canceled)

34: The process according to claim 1, which comprises the additional step of decoloring the liquid by means of a decolorizer.

35. (canceled)

36: The process according to claim 1, which comprises the additional step of regulating the pH of the liquid by means of a pH regulator.

37. (canceled)

38: The process according to claim 1, which comprises the additional step of concentrating the liquid by means of a concentrator.

39. (canceled)

40: The process according to claim 1, which comprises the additional step of desalting the liquid by means of a desalter.

41. (canceled)

42: An apparatus for performing the process according to claim 1, comprising the following components in liquid flow communication:
(I) a reactor R₁ which comprises an enzyme capable of converting educt saccharide to product saccharide;
(II) a first chromatography unit after reactor R₁ for separating product saccharide from educt saccharide; and
(III) a reactor R₂ after the first chromatography unit, wherein the reactor R₂ also comprises an enzyme capable of converting educt saccharide to product saccharide.

43-46. (canceled)

47: The apparatus according to claim 42, which additionally comprises in liquid flow communication:
means for recirculating educt saccharide to the reactor R₁; and/or
a filter; and/or
a decolorizer; and/or
a pH regulator; and/or
a concentrator; and/or
a desalter.

48. (canceled)
